(19) United States
(12) Patent Application Publication
(10) Pub. No.: US 2026/0216253 A1
(43) Pub. Date: Jul. 30, 2026

(54) A method of preventing and treating disease with transformed microbes

(71) Applicant: George Mbella Ekema, Akron, OH (US)

(72) Inventor: George Mbella Ekema, Akron, OH (US)

(21) Appl. No.: 19/125,307

(22) PCT Filed: Nov. 4, 2022
(86) PCT No.: PCT/US2022/049050

§ 371 Date: Feb. 24, 2026

(51) Int. Cl. A61K 35/66; A61K 39/00; C12N 15/63

(52) U.S. Cl. CPC A61K 35/66; C12N 15/63; A61K 39/00

(57) ABSTRACT

The covid-19 coronavirus has killed more than 5,129,829 people, globally, out of about 255,098,687 infected. About 10 million persons died from cancer in 2020, with 19.3 new cases diagnosed. About 409,000 persons died of malaria in 2019. And none of these diseases made the WHO's list of top 5 deadly diseases of 2019. Modern medicine has made tremendous progress in the prevention and treatment of disease, but the crashing of the covid-19 coronavirus and the extremely high burden of the top deadly diseases point to the urgency of radical and totally disrupting inventions in medicine. Indeed, to effectively overcome the current burden of deadly diseases we need an agile platform with exponentially better efficacy and predictability. The world is in dire need of a new medicine platform that totally and radically disrupts the current healthcare systems and the traditional ways of preventing and treating disease. The present invention does that.

The present invention uses transformed microbes, targeted to one or more mucosal surface, to prevent and treat just about any disease of importance. The current means of disease prevention relies on vaccines. As we have seen from countless failures and booster shots, vaccines have extreme limitations and increasingly fewer following. Firstly, vaccine development is unpredictable, lengthy and costly. An important lesson of the covid-19 pandemic is that waiting for several months or years to develop a vaccine, while a disease ravages the population, is unacceptable. Also, protection that is dependent on a vaccine and frequent booster shots can only find a market in despair. We have also seen complete failure of vaccines in a lot of important diseases such as HIV and cancers. The current invention presents a method of preventing disease using transformed microbes. It is more effective than vaccines because it is delivering significantly more antigens, than vaccines, in their native conformation, directly to antigen presenting cells of the immune system. It takes advantage of the natural design of the immune system, to send the messengers that the immune system pays the closest attention to (microbes), to deliver a clear and detailed message (relatively large amounts of antigens in their native conformation) to the proper receiving cells of the immune system (antigen presenting cells). This method not only prevents disease, it cures disease.

The current means of treating diseases relies heavily on medications and surgical intervention. The costs and adverse effects of drugs and the primitivity of surgical intervention are self-evident limitations of “modern medicine”. The current invention presents a method of treating disease using transformed microbes. Pathogen peptides, cancer neoantigens, and other therapeutic targets are transformed in microbes in such a manner that the microbes express the gene products in a manner as to target a specific therapeutic pathway. For example, the genes encoding all lung cancer neoantigens are transformed into a microbe, as chimeric proteins with surface antigens of an endemic pathogen for which the immune system is likely primed, in a manner that expresses the gene product in the microbial membrane. The microbes with their transmembrane trough of antigens are introduced to one or more mucosal surface, e.g., the mucosae of the GI tract, vagina, or respiratory tract. No needles. No shots. No scary tales.

It is a hundred percent about guiding the body to prevent and treat diseases, by itself, the way nature intended, with bacteria and yeasts wearing the white coats. Developing transformed microbes can take days to weeks, compared to the years and decades that it takes to develop vaccines and small molecule drugs. Transformed microbes cost a lot less to develop and can be manufactured anywhere in the world in a matter of days. The current invention will greatly reduce or eliminate the need for several diagnostic and medical procedures, vaccines, and drugs. It will save lives, ameliorate pain and suffering from disease and change economies—without a single shot.

### FIELD OF THE INVENTION

The present invention relates to a method of preventing, treating and diagnosing infectious and non-infectious diseases, including metabolic diseases, obesity and aging, by introducing microbes, that have been transformed to express a specific gene(s), to one or more of the mucosal linings, skin, and other dwelling places of microbiota.

### BRIEF DISCUSSION OF THE RELATED ART

Life is only possible because of the microbiota—the bacteria, fungi, protozoa and viruses that inhabit most of the surface area of the body. Although most abundant in the GI tract, microbiota are present is practically all mucosae and the skin. The microbiota appears to affect all systems. GI tract microbiota, for example, is known to affect the nervous system, immune system, endocrine systems, respiratory system, metabolic pathways, etc. It has even been shown that there is a direct bidirectional axis between the GI tract and lung microbiota. [1] This suggests that changes in the GI tract could affect the lungs and vice versa. The lungs are only sparsely populated with microbiota but they are in direct axis with the GI tract which is densely populated with microbiota. Indeed, there are 10 times more microbiota in the GI tract than there are cells in the human body. The genomic size of the GI tract microbiota is 100-fold greater than that of the body. Some functions of the microbiota are well known while others are just beginning to come to light. For example, they break down complex polysaccharides in the GI tract [2] and modulate the immune system. [3] Dysbiosis of the GI tract microbiota may result in a variety of illnesses of the GI tract and other systems such as cardiovascular, metabolic, nervous, etc. [4-6] And, as stated above, it has been shown recently that GI microbiota have a powerful and direct influence on the lungs. The relationship between the GI microbiota and the lungs is referred to as the “gut-lung axis”. It is believed to be a bidirectional axis. Distinct populations of microbiota inhabit the lungs and exert influence on distant sites—at least the GI tract. [7] Importantly, it now appears that chronic lung diseases, including cancer, are linked to dysbiotic lung microbiota and disorders of the GI tract. [8, 9] Also, there is a link between irritable bowel syndrome and lung disease. [10] These findings suggest that the “gut-lung axis” is indeed bidirectional. This background uses the “gut-lung axis” and the interaction between GI tract microbiota and the immune system as an example of the role of microbiota in health and disease.

### GI Tract Microbiota

There are over 1000 species of bacteria in the normal GI tract. The seeding of GI flora occurs in the first couple of years of life. After the age of two, healthy persons typically have the normal flora that they will carry into the rest of their lives. The healthy GI flora can be described as “normal” because it is quite similar in healthy persons. Most of the GI flora, more than 90%, is composed of Firmicutes and Bacteriodetes. Actinobacteria, Proteobacteria and Verrucomicrobia form the majority of the lesser phyla. [11] Around 60 species of bacteria, mostly from the Bacteroides, Bifidobacterium, Eubacterium, Faecalibacterium, Ruminococcus, and a few other genera have been noted as the “core” GI microbiota.

Microbiota are a key component of the host mucosal immunity. The microbiota, especially the GI tract microbiota, is now known to be necessary for the proper development and functioning of the immune system. [6, 26] The microbes are an abundant source of microorganism-associated molecular patterns (MAMPs) and pathogen-associated molecular patterns (PAMPs). Pattern recognition receptors (PRRs), including toll-like receptors (TLRs) and nucleotide-binding receptors (NODs), recognize MAMPs and PAMPs. [27] TLRs are a type of PRR that are used to identify domains that are shared by pathogens but distinguishable from host domains. They are highly conserved, expressed in both vertebrates and invertebrates, which suggests that they are an ancient component of the immune system. TLRs are also expressed in the mammalian nervous system. The binding of MAMPs and PAMPs to TLRs triggers different effects that are determined by the type of cell, the type of ligand and the type of TLR. TLRs are in direct contact with the lumen of the GI tract. These include TLRs expressed by intestinal epithelial cells (IECs) and TLRs expressed by immune cells, including B cells, dendritic cells, macrophages, stromal cells and T cells, in the lamina propria. TLR activation in IECs results in proliferation of epithelial cells, secretion of antimicrobial peptides and secretion of immunoglobulin A (IgA) by plasma cells in the lamina propria. [23] Some TLRs are inhibited by toll-interacting protein (TOLLIP) in IECs in response to TLR binding in the luminal membrane. NOD-like receptors (NLRs) are equivalent to TLRs but they are expressed in the cytoplasm where they bind PAMPs that enter the cell. NLRs are most valuable in cells and tissues, such the IECs, where TLR expression is downregulated to prevent overstimulation. [28]

Gut microbes enter the intestinal lamina propria through openings in the barrier or by active sampling of professional antigen presenting cells (DCs, B cells and macrophages). Once in the lamina propria, the microbes are processed by professional antigen presenting cells. [29]Normally, this results in, amongst others, the upregulation of interleukin 6 (IL-6) and subsequent differentiation of T-helper-IL-17-producing (Th17) cells. The Th17 cells trigger the activation, recruitment and migration of neutrophils by secreting IL-17A and IL-17F. [30] GI bacteria-bearing DCs induce the production of secretory immunoglobulin A (sIgA) in plasma cells. [31] The sIgA is distributed across mucosal surfaces by activated mucosal cells. The GI microbes also induce the expression of factors leading to the induction of IgA+ B cells. [32] This priming of the immune system by the GI tract microbiota makes the immune system ready for a rapid and effective response when required. Some populations of GI tract microbiota, for example Bacteroides fragilis, Bifidobacterium infantis, and Clostridium clusters IV and XIVa, induce regulatory T cells (Tregs) that induce the secretion of anti-inflammatory cytokine IL-10 to counterbalance the effects of the proinflammatory Th17. [34]

The effects of these microbiota, even single populations, on the immune system is momentous. For example, individual members of Bifidobacterium are capable of inducing human peripheral blood mononuclear cells to mature into DCs. [35] Also, Bifidobacterium lactis increased the proportion of mononuclear leukocytes, phagocytic capacity and tumoricidal activity of NK cells in healthy adult volunteers. [36]

Some bacteria affect the immune system indirectly by secreting products such as short-chain fatty acids. Both endothelial cells and leukocytes have receptors for SCFAs. [40] It is clear that microbes of the microbiota, and others, affect the immune system and other systems at the organismal level. They also affect these systems by their products. For example, short-chain fatty acids (SCFAs) produced by bacteria as a by-product of fermentation of dietary fiber. Some of the prolific producers of SCFA include Bacteroides, Bifidobacterium, Clostridium, Eubacterium, Lactobacillus, Prevotella, Propionibacterium, and Roseburia.[37] SCFA production is beneficial in increasing the acidity of the intestinal lumen which renders it unfavorable for pathogens. [38] More importantly, SCFAs have receptors, GPR41, GPR43 and GPR109a, on leukocytes and endothelial cells. [40, 41] GPR109a is butyrate-specific and triggers anti-inflammatory pathways. [41] Major butyrate producers include the Firmicutes with acetyl-CoA pathway genes. Butyrate is the primary energy source for the intestinal epithelium and plays a role in barrier integrity. [42, 43] It is selectively transported into the colon epithelium where it triggers activation, proliferation and migration of immune cells, cell adhesion, secretion of cytokines and apoptosis of cancer cells. [26, 41]. Its primary mechanism is the inhibition of histone deacetylase (HDAC). The inhibition of HDAC affects the acetylation of histones, such as Treg FoxP3 locus—which is vital for Treg maturation, and of major transcription factors, such as nuclear factor kappa-light-chain-enhancer for activated B cells (NF-κB) and signal transducer and activator of transcription 3(Stat 3). [44-46]

A balanced microbiota is vital for the proper functioning of the immune system and other systems. It has been demonstrated, in humans and animals, that dysbiosis has effects that are detrimental to health. For example, germ-free mice have an impaired immune system. They have relatively small Peyer's patches, fewer CD8αβ intraepithelial lymphocytes, and relatively underdeveloped isolated lymphoid follicles. They also lack primed T cells, have impaired production of mucosal IgA antibodies, and active IL-10-mediated inflammatory hyporesponsiveness. [51,52] Another example is dysbiotic mice with colitis-associated cancer (CAC). These mice are unable to process pro-IL-1β and pro-IL-18 which results in a greater tumor burden. [53]

### Microbiota and the Immune System Versus Cancer

Cancer cells express neoantigens. These neoantigens are specific to each type of cancer. The neoantigens distinguish cancer cells from neighboring healthy cells. The immune system takes advantage of these neoantigens and goes after cancer cells in a process that has been described as the “cancer immunity cycle”. [54] The first step of the cancer immunity cycle is the capture of neoantigens on cancer cells by DCs. This capture alone is insufficient to trigger an immune response against the cancer cells. For an immune response against the cancer cells to occur, a secondary signal is necessary. The secondary signal could come from microbiota, proinflammatory cytokines, or factor released from dying cancer cells. Lack of proper signals would result in peripheral tolerance to the neoantigens.

In the first step of the cancer immunity cycle DCs capture neoantigens expressed by cancer cells. They process the captured neoantigens and present them to T cells. The T cells are primed and activated into effector T cells targeted to the cancer neoantigens. The effector T cells migrate to, and invade, the tumor bed where they bind to the neoantigens and kill cancer cells. The nature and extent of the immune response is determined by the balance between the effector T and regulatory T cells. If the neoantigens are not detected, that is DCs and T cells treat the neoantigens as “self”, it results in a Treg response rather than an effector T response. Also, another situation could arise where DCs capture the neoantigens and T cells are activated but the effector T cells are unable to infiltrate the tumor bed or are inhibited by factors in the tumor microenvironment. There are two main inhibitors of T cell response. The first major inhibitor of T cell response is programmed cell death ligand 1 (PD-L1). PD-L1 is expressed on tumor cells and on tumor-infiltrating immune cells. It binds programmed cell death protein 1 (PD-1) on effector CD8+ T cells and blocks the production and secretion of the cytotoxic factors needed to kill tumor cells. The second major inhibitor of T cell response is cytotoxic T lymphocyte-associated protein 4 (CTLA-4). CTLA-4 is expressed on Tregs. It inhibits the priming and activation of effector CD8+ T cells by binding DC80 and CD86 ligands on APCs.

These inhibitors have undermined immune-based cancer therapies so novel therapies are using anti-PD-L1 and anti-CTLA-4 antibodies to suppress the inhibitors. The hope is that, by suppressing the inhibitors, the cancer immunity cycle will progress, unhindered, until all of the cancer cells are killed. Whether or not these therapies are effective, it is clear that the immune system's peripheral tolerance must be suppressed for a more robust response to neoantigens by DCs and T cells.

Microbiota induce the generation of CD4+ T cells against their own antigens thereby limiting the systematic dissemination of microbiota. [55, 56] The same effect of antigen cross-reactivity, or superantigen-driven response, accounts for T cell-dependent tumor regression. Studies in mice have shown that Th17 cells and memory Th1 cells elicited against commensal bacteria might preferentially accumulate in an inflammatory tumor microenvironment, already primed by bacterial products or ligands for PRRs. [47, 57, 58] The long-range effect of microbiota has been explained by two signal hypotheses. [59] Signal 1 hypothesis suggests antigen mimicry or cross reactivity. That is, certain microbial antigens from the bacterial species that infiltrate the lamina propria closely resemble tumor antigens. When these bacterial antigens are used to prime T cells there is a more robust and effective anti-tumor immune response. [59] This hypothesis makes sense in light of the fact that there are essentially limitless antigens in the microbiota and the likelihood that immunosurveillance would necessitate the suppression of immune inhibitory factors. Signal 2 hypothesis holds that by interacting with PRRs after passing the intestinal barrier, microbiota can stimulate the production of a diverse palette of cytokines and interferons and determine whether it will elicit a proinflammatory, immunostimulatory, or immunosuppressive response. [59] There is also evidence that commensal-specific Tregs can switch to effector inflammatory Th17 cells following disruption of the mucosal barrier. This local response builds into a systemic response that targets the cancer.

Evidence of a systemic anti-cancer response from the GI microbiota has been reported, for example, by Gui et al. [60] They studied the effect of GI microbiota on lung cancer using the Lewis lung cancer mouse model. The objective was to study the effect on tumor size of a depleted GI microbiota and a supplemented microbiota. One group of mice were treated with an antibiotic cocktail of vancomycin, ampicillin, and neomycin plus cisplatin. A second group of mice were treated with Lactobacillus bacteria and cisplatin. The tumor burden was larger, and the survival rate lower, in mice treated with the antibiotic cocktail and cisplatin. On the other hand, the tumor burden was lower, and the survival rates were higher in mice treated with Lactobacillus and cisplatin. So, it supports the findings of other studies that dysbiosis of the microbiota results in a greater tumor burden; whereas, a healthy microbiota results in a lesser tumor burden and increased survival. Additionally, gene expression studies suggest that dysbiosis of the microbiota can partially impair the effect of cisplatin by upregulating the expression of vascular endothelial growth factor A (VEGFA) and downregulating the expression of Bcl-2-associated x protein (BAX) and cyclin-dependent kinase inhibitor 1B (CDKN1B). Also, the dysbiosis of microbiota downregulated the expression of interferon gamma (IFN-γ), granzyme B (GZMB), and perforin 1(PRF1) in the CD8⁺ T cells. It appears, therefore, that healthy GI microbiota can contribute to the anti-lung cancer response by enhancing the antigrowth and proapoptotic effects of cisplatin.

### Vaccination

Vaccination involves the delivery, typically by injection, of noninfectious antigen(s) from known pathogens, along with an adjuvant, to achieve immunization. The concept of vaccination relies on immunological memory. The effect of vaccination is to elicit immunological memory, and thus protection from infection to specific pathogens. The concept of vaccination, especially by injection, is unnatural and frequently ineffective as a reliable means of immunization. Vaccine development can take years, cost millions, and still provide marginal of no immunization. There are too many limitations of injected vaccination. For example, injected vaccination stimulates the systemic immune system, eliciting humoral and cell-mediated immunity, but has little effect on the mucosal immune response, which presents a challenge because many pathogens are deposited and replicate in mucosal compartments, and the injection does not provide the most efficient immune memory for these disease agents. For this reason, vaccine developers are interested in developing new vaccines that are delivered unto mucous membranes or applied transcutaneously. Vaccines administered in the mucosa elicit both mucosal and systemic immunity and produce the same level of protection from disease as injected vaccines. Currently, a version of intranasal influenza vaccine is available, and the polio and typhoid vaccines can be administered orally. Also, measles and rubella vaccines are being adapted to aerosol delivery using inhalation devices.

### Intestinal Antigen Presenting Cells

The GI tract is not a free for all for microbes. The microbiota and other microbes that find their way into the GI tract are carefully and fully evaluated and monitored by the immune system —mainly the antigen presenting cells (APCs). The GI tract APCs, typically DCs and macrophages, integrate signals from the microenvironment to orchestrate innate and adaptive immune responses that ultimately lead to durable tolerance of the microbiota. Tolerance is not a default response, however, because macrophages and DCs remain poised to vigorously respond to pathogens that breach the epithelial barrier. [61] So, the immunity governing the GI microbiota is not just about memory but also active surveillance.

The GI microbiota is enormous, diverse, and complex. It presents an immense antigenic challenge that has the potential to trigger vast intestinal inflammation. Normally, there is no inflammation because the body maintains homeostasis via a sophisticated immune network that affords tolerance to the microbiota while promoting responsiveness to invading pathogens. [62, 63] The APCs, predominantly DCs and macrophages, are central to this discrimination process. They are separated from the microbiota by a single layer of epithelial cells. The APCs integrate cues from epithelial, immune, and stromal cells to direct innate and adaptive immunity. [64-70] Inappropriate responses to these signals can lead to a breakdown of tolerance toward the microbiota and result in uncontrolled inflammation, such as that observed in Crohn's disease and ulcerative colitis. [71] The tissue microenvironment regulates the differentiation of macrophages and DCs from myeloid progenitor cells. The local intestinal milieu is shaped by microbiota, enteric antigens, and immune cells that collectively contribute to the developmental outcome of GI APCs. For example, intestinal macrophages are maintained and replenished by Ly6C⁺ monocytes in the “monocyte waterfall”. The Ly6C⁺ monocytes differentiate into resident intestinal macrophages through a series of intermediary stages. [72-75] The monocytes that produce intestinal macrophages are derived from macrophage-DC progenitors—the same bone marrow progenitors capable of producing intestinal DCs. [76] The outcome of the macrophage-DC progenitors is affected by specific cytokines and growth factors in the tissue microenvironment. For example, colony stimulating factor 1 (Csf1) receptor controls the maturation of monocytes that produce intestinal macrophages. So, Csf1 receptor-deficient mice and mice treated with anti-Csf1 receptor antibody have lower numbers of intestinal macrophages as are Csf1op/op mice, which have a mutation in the gene encoding Csf1. [77-79]

### GI Epithelial Cell Mch Class II Expression and Antigen Presentation

The epithelial cells of the GI tract, lungs, and very likely all mucosae, express class II major histocompatibility complex (MHC class II) molecules. The expression of MHC class II molecules on epithelial cells suggests that these cells may function as APCs—actually are APCs. Epithelial cells form the barrier between an organism and its environment so it should not be surprising that these cells have the capacity to affect a balance between tolerance of the microbiota and immunity against pathogenic microbes. Afterall, it is at the epithelial surface that the first interaction between an organism and microbes occur.

The adult small intestine is about 5 meters long. Its epithelium is arranged into villi and crypts of Lieberkühn which drastically increase the surface area. The large intestine comprises the distal 1.5 meters of the GI tract. In general, the intestinal mucosa is a vast, 200-300 m², layer of simple columnar epithelium. [80] This vast surface facilitates the absorption of nutrients and water, and modulates the immune system. [81] There are many types of intestinal epithelial cells (IECs), with different functions, including enterocytes, enteroendocrine cells, goblet cells, M cells, Paneth cells and tuft cells. Enterocytes are the most numerous of the IECs. They absorb nutrients and transcytose antimicrobial proteins and IgA. [82] Enteroendocrine cells secrete neurohormones such as gastric inhibitory peptide, glucagon-like peptide, and vasoactive intestinal peptide. [82] Goblet cells secrete mucus and trefoil factor. They also secret resistin-like molecule b, which modifies T cell-mediated immunity. They also deliver antigens to submucosal DCs through specialized antigen passages. [83, 84] Paneth cells sustain stem cells to promote intestinal regeneration and they secrete the antimicrobial proteins a-defensins, C type lectins, lysozyme and phospholipase A2. [6] M cells transcytose antigens to the underlying gut-associated lymphoid tissue (GALT)—a collection of intraepithelial lymphocytes (IELs) and lamina propria lymphocytes (LPLs). [85-87] Tuft cells are chemosensory and may play a role in type 2 immunity and mucosal immunity. [88, 89] IECs affect innate immunity with a variety of pattern recognition receptors (PRRs). Activation of PRRs results in the release of cytokines and chemokines. PRRs, including Toll-like receptors 1-9 (TLRs) and nucleotide-binding oligomerization domain-containing proteins (NODs), recognize pathogen-associated molecular patterns from microbes. [90-92] In vitro, the apical TLRs (TLR1 and TLR2), that are expressed on the luminal aspect of the intestines, appear to be hyporesponsive to PAMPs, whereas PRRs that are expressed in the cytoplasm (NOD1 and NOD2), endosomes (TLR3, TLR4, TLR7, TLR8 and TLR9), submucosal basolateral membrane (TLR4 and TLR5) respond ‘normally’. [92-94] This observation is consistent with the immune tolerance of the GI microbiome.

### MHC Class II and Costimulatory Molecules

MHC class II molecules are transmembrane ab heterodimers. There are three MHC class II isotypes in humans—HLA-DR, HLA-DP, and HLA-DQ. They are encoded by a and b chain genes within the Human Leukocyte Antigen (HLA) locus on chromosome 6. Class II transactivator (CIITA) tightly regulates the MHC class II expression and function by recruiting DNA-binding factors, chromatin-modifying proteins, and transcription initiators to the MCH class II locus. MCH class II molecules process and present antigens in a complex manner that involves accessory molecules and trafficking through intracellular compartments. [95-97] Nascent MHC class II molecules in the ER are chaperoned by invariant chain (CD74) into the MCH II compartment (MIIC)—a low-pH, late-stage endosomal compartment. In the MIIC, CD74 is cleaved by proteases into class II invariant chain-associated peptides (CLIP). [97] MHC class II binds CLIP temporarily. HLA-DM, a catalytic protein, swaps CLIP for peptides that bind MHC class II molecules with a higher affinity. [98] The peptide-MHC class II complex is trafficked to the cell surface so that it can interact with CD4+ T cells. Certain APCs, such as B cells, certain DCs, and thymic epithelial cells, express HLA-DO which regulates HLA-DM and competitively inhibits the DM-MCH class II interaction. [99]

The interaction of peptide-MCH class II and CD4+ T cells is the first signal to trigger an antigen-specific adaptive response in the lymphocytes. A second signal is required for the efficient activation of naïve CD4+ T cells. That is, the APC-T cell interaction is co-stimulated by signals such as CD80, CD86, and the B7 family that interact with stimulatory CD28 or inhibitory CTLA-4 on T cells. [100] In professional APCs, these molecules are upregulated in response to PAMPs and damage-associated molecular patterns (DAMPs), such as ATP. [101] A hyporesponsive, anergic state, results in T cells when there is insufficient co-stimulation of the peptide-MHC class II interaction. [102]

### Role of IECS in Antigen Presentation

All segments of the small intestines express MCH class II, HLA-DM, and CD74. [103-108] Expression starts as early as 18 weeks of gestation and increases with fetal development. [109-111] Normally, MHC class II molecules are expressed on the enterocytes in the upper villus and diminishes to nothing in the crypts. [107, 109, 112] There is no expression of MHC class II in colonic epithelium under normal circumstances, but expression has been noted in pathological states such as celiac disease, graft versus host disease, and inflammatory bowel disease (IBD). [106, 113-119] In active celiac disease, exposure to inflammatory antigens increases the expression of surface MHC class II molecules. [115, 120, 121] IECs are polarized. Enzymes are localized in the apical surface to digest dietary antigens and poly-Ig receptors are localized in the basolateral membrane to translocate IgA into the intestinal lumen. [122] IEC polarity is essential for peptide presentation to the resident immune cells of the GALT, which, in turn, is necessary for systemic crosstalk. Several in vitro studies have shown both apical and basolateral expression of MHC class II in IECs. [106, 112,123-126] In vivo studies have found MHC class II expression of physiological relevance in the basolateral membrane. [127-129] MARCH 8 ubiquitin ligase internalizes MHC class II molecules in IECs. It may have to be downregulated for robust surface expression of MCH class II. [130] A similar mechanism has been observed in DCs, where MARCH 1is downregulated during TLR ligand-induced maturation. [131]

### Lung Microbiota

The microbiota of the lungs and airways are distinct from those of other mucosae. [132] Additionally, the lower respiratory tract microbiome is distinct from that of the upper respiratory tract. [133] Firmicutes and Actinobacteria are predominant in the nostrils and Firmicutes, Proteobacteria and Bacteroidetes are predominant in the oropharynx. [13] The lung has mostly Bacteroidetes, Firmicutes and Proteobacteria. The nasal communities resemble skin bacteria and do not contribute much to the lung communities. [134] There is a dysbiosis of lung microbiota in chronic obstructive pulmonary disease (COPD) and lung cancer. [7, 19] When the lung is inflamed, the epithelium losses integrity and serum proteins leak into the airways. [135] Neutrophils and monocytes from alveoli are attracted by formyl peptides and bacterial products such as cleavage peptides. [136] The emigration of neutrophils is vital for defense against pathogens and for fighting tumors, however, the influx and degranulation of neutrophils in the airways and parenchyma of the lungs results in chronic inflammation, progressive small airway obstruction and parenchymal lung damage. [137, 138] When the lumina are obstructed with mucus, areas of increased temperature and decreased oxygen tension arise. The altered temperature and oxygenation in these areas trigger dysbiosis of the lung microbiome. The normal Bacteroides phylum is often replaced with Proteobacteria, such as Pseudomonas aeruginosa, Haemophilus influenza, and Moraxella catarrhalis, and Firmicutes, such as Streptococcus pneumoniae and Staphylococcus aureus.[17, 19, 20, 139, 140] Dysbiosis of the lung microbiome has also been observed with the generation of intra-alveolar catecholamines and inflammatory cytokines. [141] IL-6 and IL-8 are elevated during inflammatory stress. They promote tumorigenesis by stimulating the NF-κB-1 pathway in epithelial cells. [142] They are also expressed by premalignant and senescent lung cancer cells and may act in an autocrine and/or paracrine manner to promote the proliferation, migration and invasion of cancer cells. [143-145]

### Bidirectional Concept of the GI Tract-Lung Axis

There is an interconnected axis between the lungs and the GI tract and it is reliant on microbiota. This is also true for other parts of the body, e.g., the axis of the oral cavity and cardiovascular system. In the case of the GI tract-lung axis, ingested microbes can access both the lungs and the GI tract. [6, 146] Translocated microbes in the lamina propria, and pieces thereof, are transferred to the mesenteric lymph nodes, by APCs, where they are used to prime naïve B and T lymphocytes. In addition to producing immunoglobulins in situ, activated B lymphocytes migrate to draining lymph nodes and other mucosae. Steady state antigen influx stimulates inflammasome conversion of pro-IL-1β and pro-IL-18 into active form. This conversion inhibits the production of IL-10 and other anti-inflammatory molecules and facilitates the migration of DCs to local lymph nodes and the priming and differentiation of T lymphocytes. The T lymphocytes migrate from the GALT to mucosal and non-mucosal peripheral tissues, including the bronchial epithelium. [147-149] This mechanism also works in reverse—i.e, from the lungs to the GI tract. In fact, it is reasonable to assume that all mucosae are interconnected by similar axes and that microbes can mediate a systemic immune response from virtually any mucosa—distant or far, peripheral or deep.

### Influence of the Microbiota on Lung Health

Microbiota affects the health of the lungs. Germ-free (GF) mice, with compromised GI and lung microbiota, have impaired pathogen clearance in their lungs. [52] The composition of “healthy,” or rather balanced, gut microbiota is shown to have a serious influence on the effectiveness of lung immunity. GF mice, devoid of their intestinal microbiota during the development of their immune system, show impaired pathogen clearance in the lung, which results in their growth and dissemination [52]. It has also been shown that increased use of penicillins, cephalosporins, macrolides, and quinolones correlated with an increased risk of lung cancer in humans [150]. Also, obese mice with dysbiosis of the GI microbiota have an impaired expression of the cytokines IFNα, IFNβ, IL-6, and TNFα in their lungs. They also have a decreased expression of IFNγ, interleukin 2 receptor subunit beta (IL-2RB), and perforin 1 (Prf1). These deficiencies are reversible with a daily supplementation of probiotic strain of Lactobacillus gasseri. [151]

### Probiotics and Health

Probiotics are microbes that are believed to provide a health benefit when ingested or applied. [152] They are mostly for the GI tract and mainly from the Lactobacillus and Bifidobacterium genera—with many strains, including B. animalis subsp. Lactis, B. bifidum, B. breve, B. longum, L. acidophilus, L. fermentum, L. johnsonii, L. paracasei, L. plantarum, L. reuteri, and L. rhamnosus. There is great genetic variability between and within the different genera. [153] This genetic diversity probably provides a myriad of antigens from which most of the immune system is built. However, the large burden of disease indicates that more antigens are necessary. For probiotics to be effective at preventing or treating the current disease burden, we would require thousands of genera and billions of strains and hope that, upon consuming the mountain of microbes, the large volume will provide the required antigens. Or, we could just express these antigens in microbes. These additional antigens can be artificially expressed in microbes that are considered probiotics, symbiotics, or any type of microbe—and that's the subject of this invention. With this technology a very large number of antigens can be expressed in a single strain of bacteria or yeast. For example, more effective versions of all vaccines, ever made, can be expressed in a single strain of bacteria or yeast and reduced to a single pill. Yes, the current invention can replace all vaccines with a single pill, and the pill would provide a vastly better efficacy and safety profile than vaccination. Similarly, by expressing multiple neoantigens in a single strain of bacteria or yeast, a single pill could treat and prevent multiple types of cancer. Artificially expressing antigens in microbes and distributing these microbes to the various mucosa, skin, etc., has the potential of disrupting global healthcare systems by providing rapid and highly effective prevention and treatment of most of the troubling diseases at a fraction of current healthcare costs.

This background is intended to provide a fundamental understanding of the essence of the microbiome. It does not cover the entire scope of plant and animal microbiome of which is relevant to this patent application. Other background sources, e.g. Elsevier's ‘The Microbiome, Vol 176’(ISBN: 9780128207970 [hardcover], ISBN: 9780128207987 [eBook]—and herein incorporated by reference) may provide insight into additional microbiome axes, such as the GI tract-brain axis.

### Molecular Cloning and Microbial Fermentation

The techniques of molecular cloning, microbial fermentation, and biotechnology, in general, required for this invention, are well known in the art. A person of average skill in the art is familiar with the technology. Some exemplary references are listed below and are hereby incorporated by reference in their entirety.

### Manual of Industrial Microbiology and Biotechnology by Richard H. Baltz, Arnold L. Demain and Julian E. Davies.

### Molecular Biotechnology: Principles and Applications of Recombinant DNA by Bernard R. Glick, Jack J. Pasternak and Cheryl L. Patten.

### Principles of Fermentation Technology by Peter F Stanbury, Allan Whitaker and Stephen J Hall.

### Biotechnology: A Textbook of Industrial Microbiology by Wulf Crueger.

### Formulation and Dosing

Formulation and dosing are consistent with current methods of probiotics formulations and dosing. Additionally, some embodiments use microbial products, such as fragments of microbes bearing target peptides in pieces of membrane. Standard methods such as those described by Fenster et al., are well established and wildly known in the art. The work below is hereby incorporated by reference in its entirety.

Kurt Fenster, Barbara Freeburg, Chris Hollard, Connie Wong, Rune Rønhave Laursen, and Arthur C. Ouwehand. The Production and Delivery of Probiotics: A Review of a Practical Approach, Microorganisms, 2019 March; 7(3): 83

### SUMMARY OF THE INVENTION

If humans and animals had a microbiome that included all of the millions of beneficial microbes found in nature, then it would be very likely that such humans and animals would be resistant to just about every known disease. But since we actually have only a tiny fraction of microbial species, we can achieve the same fit by altering microbes to express specific gene(s) and gene products. For example, we can express a limitless array of antigens—say, for infectious diseases and cancer—and use the microbes or their products to deliver these antigens to APCs in the mucosae (e.g. GI tract) and other pertinent sites.

It is an objective of this invention to present a method to prevent infectious diseases, in humans and animals, by transforming microbes, typically bacteria and fungi (but even possibly protozoa and viruses—plant cells in some cases), with genes encoding antigens of one or more infectious disease agent in a manner that causes the transformed microbe to express the antigens. The transformed microbe is then distributed over a body surface(s), including the skin, vagina, GI tract, and airways. The transformed microbe may also be applied transdermally.

It is an objective of this invention to present a method to treat infectious diseases, in humans and animals, by transforming microbes, typically bacteria and fungi (but even possibly protozoa and viruses—plant cells in some cases), with genes encoding antigens of one or more infectious disease agent in a manner that causes the transformed microbe to express the antigens. The transformed microbe is then distributed over a body surface(s), including the skin, vagina, GI tract, and airways. The transformed microbe may also be applied transdermally.

It is an objective of this invention to present a method to prevent non-infectious diseases, such as cancers, in humans and animals, by transforming microbes, typically bacteria and fungi (but even possibly protozoa and viruses—plant cells in some cases), with genes encoding neoantigens of one or more cancer(s) in a manner that causes the transformed microbe to express the neoantigens. The transformed microbe is then distributed over a body surface(s), including the skin, vagina, GI tract, and airways. The transformed microbe may also be applied transdermally.

It is an objective of this invention to present a method to treat non-infectious diseases, such as cancers, in humans and animals, by transforming microbes, typically bacteria and fungi (but even possibly protozoa and viruses—plant cells in some cases), with genes encoding neoantigens of one or more cancer(s) in a manner that causes the transformed microbe to express the neoantigens. The transformed microbe is then distributed over a body surface(s), including the skin, vagina, GI tract, and airways. The transformed microbe may also be applied transdermally.

It is an objective of this invention to present a method to prevent non-infectious diseases, such as allergies and asthma, in humans and animals, by transforming microbes, typically bacteria and fungi (but even possibly protozoa and viruses—plant cells in some cases), with genes encoding antigens of one or more trigger(s) in a manner that causes the transformed microbe to express the antigens. The transformed microbe is then distributed over a body surface(s), including the skin, vagina, GI tract, and airways. The transformed microbe may also be applied transdermally.

It is an objective of this invention to present a method to treat non-infectious diseases, such as allergies and asthma, in humans and animals, by transforming microbes, typically bacteria and fungi (but even possibly protozoa and viruses—plant cells in some cases), with genes encoding antigens of one or more trigger(s) in a manner that causes the transformed microbe to express the antigens. The transformed microbe is then distributed over a body surface(s), including the skin, vagina, GI tract, and airways. The transformed microbe may also be applied transdermally.

It is an objective of this invention to present a method to treat autoimmune diseases, in humans and animals, by transforming microbes, typically bacteria and fungi (but even possibly protozoa and viruses—plant cells in some cases), with genes encoding antigens of one or more trigger(s) in a manner that causes the transformed microbe to express the antigens. The transformed microbe is then distributed over a body surface(s), including the skin, vagina, GI tract, and airways. The transformed microbe may also be applied transdermally.

It is an objective of this invention to present a method to prevent metabolic diseases and obesity, in humans and animals, by transforming microbes, typically bacteria and fungi (but even possibly protozoa and viruses—plant cells in some cases), with genes encoding peptides of one or more therapeutic targets in a manner that causes the transformed microbe to express the therapeutic target. The transformed microbe is then distributed to pertinent mucosae where expression of the therapeutic peptide will affect the desired therapeutic effect.

It is an objective of this invention to present a method to treat metabolic diseases and obesity, in humans and animals, by transforming microbes, typically bacteria and fungi (but even possibly protozoa and viruses—plant cells in some cases), with genes encoding peptides of one or more therapeutic targets in a manner that causes the transformed microbe to express the therapeutic target. The transformed microbe is then distributed to pertinent mucosae where expression of the therapeutic peptide will affect the desired therapeutic effect.

It is an objective of this invention to present a method to rejuvenate and improve the appearance of skin, and minimize or reverse the appearance of aging, in humans and animals, by transforming microbes, typically bacteria and fungi (but even possibly protozoa and viruses - plant cells in some cases), with genes encoding pertinent peptides in a manner that causes the transformed microbe to express the peptides. The transformed microbe is then distributed over a body surface(s), including the skin, vagina, GI tract, and airways. The transformed microbe may also be applied transdermally.

It is an objective of this invention to present a method to diagnose diseases, in humans and animals, by transforming microbes, typically bacteria and fungi (but even possibly protozoa and viruses—plant cells in some cases), with genes encoding probing peptide(s) that interact with known disease markers (or products thereof) in a manner that causes the transformed microbe to express the probing peptide(s). The transformed microbe is then distributed over a body surface(s), including the skin, vagina, GI tract, and airways. The transformed microbe may also be applied transdermally.

### BRIEF DESCRIPTION OF THE DRAWINGS

In order that the invention may be more readily understood, and so that further features thereof may be appreciated, the invention will now be described by way of example with reference to the accompanying drawings, in which FIGS. 1 to 5 relate to the transformation of a generic microbe, and in which:

FIG. 1 shows the transformation of a generic microbe with a vector, such as plasmid (1), with a promoter (2), target gene (3) and selection sequence(s) (4).

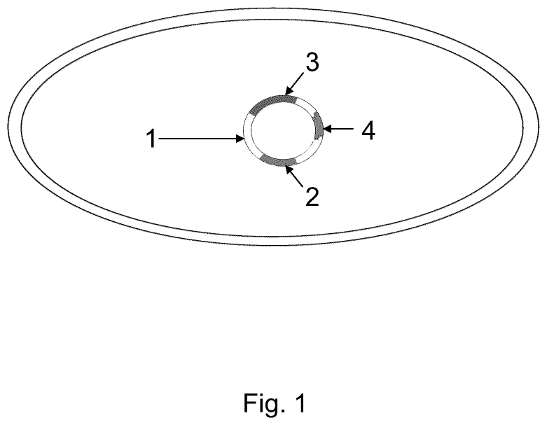

FIG. 2 shows the transformed generic microbe expressing the target gene product as a transmembrane protein with the vast majority of the sequence expressed as extracellular domains (9) with minimal intracellular domains (10) and native or designed transmembrane domains (8).

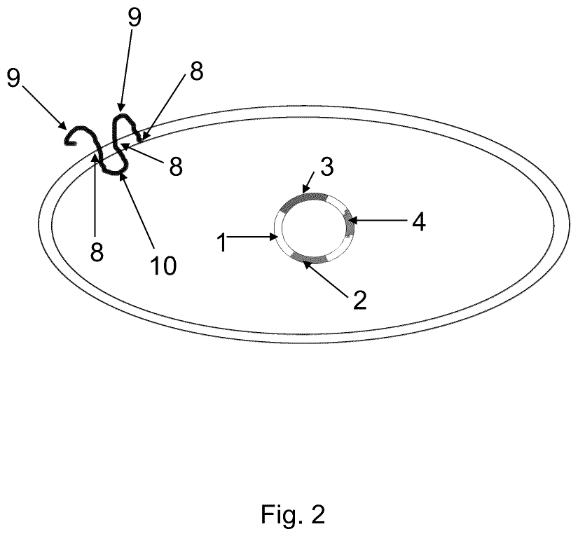

FIG. 3 shows the transformed generic microbe expressing the target gene product as a secretable peptide (5) with a signal sequence (6) guiding its secretion to the extracellular milieu (7).

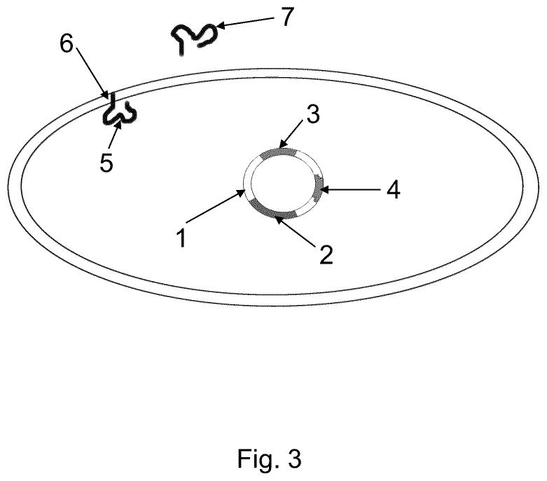

FIG. 4 shows the generic microbe transformed with multiple vectors simultaneously, with each vector encoding a distinct target gene product, for example, a neoantigen for non-small cell lung adenocarcinoma (1), a neoantigen for non-small cell lung squamous cell carcinoma (11), a neoantigen for non-small cell lung undifferentiated carcinoma (12), etc., to create an effective prevention and treatment of all lung cancers.

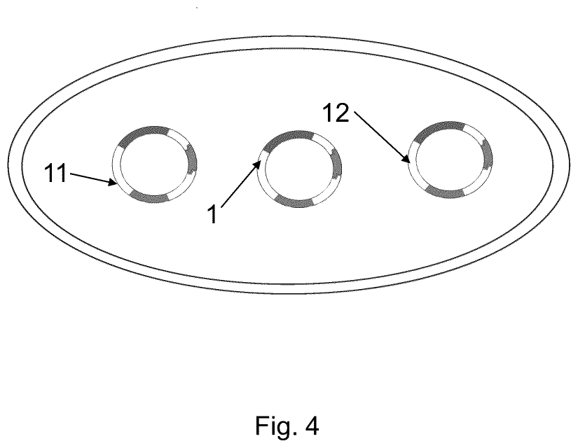

FIG. 5 shows the generic microbe transformed with a vector containing a chimeric sequence, for example, a neoantigen (14) and a surface protein of a pathogen (13)—especially of a pathogen that immune system is primed to.

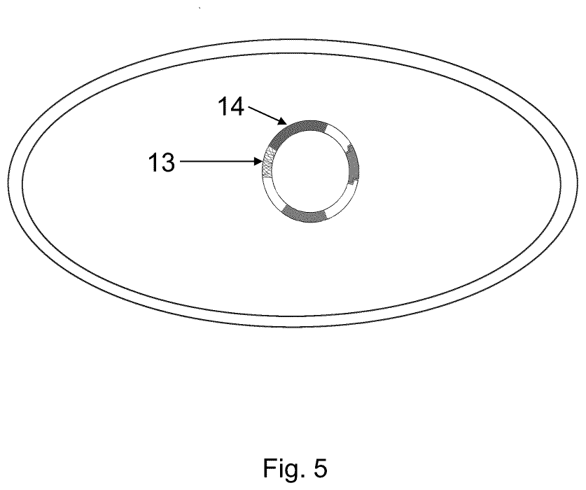

The figures accompanying this document, as well as the detailed description of embodiments, are exemplary only, and are not intended to show the bounds of the current invention. Also, the figures are not to scale.

### DETAILED DESCRIPTION OF EXEMPLARY EMBODIMENTS OF THE INVENTION

In accordance with one aspect of the present invention, is a transformed microbe, such as bacteria or yeast cells—even a plant cell in some cases. The microbes are rendered competent using standard laboratory protocols and are transformed with genetic material in a manner such that the gene product of said genetic material is expressed by the microbes. In a preferred embodiment said genetic material is reproduced with the microbe such that newly reproduced microbes have a copy of said genetic material. Such an expression and reproduction of genetic material is common knowledge to a person of average skill in the art. Preferentially, the transformation of the microbe with genetic material is achieved by means of a vector. In an exemplary embodiment, plasmid or vector transformation is used to introduce genetic material into the microbe. Plasmid or vector transformation is the process by which exogenous DNA is transferred into the host cell. Transformation usually implies uptake of DNA into bacterial, yeast or plant cells, while transfection is a term usually reserved for mammalian cells. Typically, the method for transformation of a DNA construct into a host cell is chemical transformation, electroporation or particle bombardment. In chemical transformation, cells are made competent (rendered able to take up exogenous DNA) by treatment with divalent cations such as calcium chloride, which make the bacterial cell wall more permeable to DNA. Heat shock is used to temporarily form pores in the cell membrane, allowing transfer of the exogenous DNA into the cell. In electroporation, a short electrical pulse is used to make the bacterial cell temporarily permeable. Particle bombardment, is typically used for the transformation of plant cells. Gold or tungsten particles are coated with the DNA construct and physically forced into the cell by gene gun. Protocols and reagents for transformation are readily available, commercially, and the various techniques are common knowledge to a person of average skill in the art.

In a preferred embodiment, the microbe is transformed with a piece of exogenous DNA (or gene) that is ligated to a plasmid or vector in a manner that causes expression of the exogenous DNA by the microbe. The recombinant plasmid or vector to be transfected into the microbe is created by connecting the insert DNA (exogenous DNA or gene) into a compatibly digested vector backbone. This is accomplished by covalently connecting the sugar backbone of the two DNA fragments. This reaction, called ligation, is performed by the T4 DNA ligase enzyme. The DNA ligase catalyzes the formation of covalent phosphodiester linkages, which permanently join the nucleotides together. After ligation, the insert DNA is physically attached to the backbone and the complete plasmid can be transformed into microbial cells for propagation and expression.

The majority of ligation reactions involve DNA fragments that have been generated by restriction enzyme digestion. Most restriction enzymes digest DNA asymmetrically across their recognition sequence, which results in a single stranded overhang on the digested end of the DNA fragment. The overhangs, called “sticky ends”, are what allow the vector and insert to bind to each other. When the sticky ends are compatible, meaning that the overhanging base pairs on the vector and insert are complementary, the two pieces of DNA connect and ultimately are fused by the ligation reaction.

Usually, two different restriction enzymes are used for adding an insert into a vector (one enzyme on the 5′end and a different enzyme on the 3′end). This ensures that the insert will be added in the correct orientation and prevents the vector from ligating to itself during the ligation process. If the sticky ends on either side of the vector are compatible with each other, the vector is much more likely to ligate to itself rather than to the desired insert. The various protocols and reagents for molecular cloning are widely available commercially and are common knowledge to a person of average skill in the art.

In a preferred embodiment, the exogenous DNA or gene is a DNA fragment or gene of a pathogen. To develop immunity against, or treat, a particular pathogen, a DNA fragment or gene of that pathogen is transformed into a microbe in such a manner that the microbe expresses said DNA fragment or gene. The microbe is amplified by culturing. The transformed microbe is introduced to one or more of the mucous membranes, e.g., airways, GI tract, vagina. It can also be introduced the transdermally. Several pathogen peptides can be expressed simultaneously to create a more efficient disease-preventing microbe.

In a preferred embodiment, the exogenous DNA or gene of multiple (more than one) pathogens are transformed into a microbe such that the transformed microbe will affect immunity or treatment against multiple pathogens. For example, DNA fragments and/or genes from several pathogens can be transfected into a microbe such that the microbe will affect immunity against all known sexually transmitted diseases. Multiple recombinant plasmids or vectors could be used or chimeric methods could be used with a single recombinant plasmid or vector.

In a preferred embodiment, the exogenous DNA or gene is a DNA fragment or gene encoding a cancer neoantigen or other therapeutic target selectively expressed by cancer cells. To develop immunity against, or treat, a particular cancer, a DNA fragment or gene encoding a neoantigen or a therapeutic target that is selectively expressed by the cancer is transformed into a microbe in such a manner that the microbe expresses said DNA fragment or gene. The microbe is amplified by culturing. The transformed microbe is introduced to one or more of the mucous membranes, e.g., airways, GI tract, vagina. It can also be introduced the transdermally.

In a preferred embodiment, the exogenous DNA or gene encoding neoantigens, or other therapeutic targets, of multiple (more than one) types of cancer are transformed into a microbe such that the transformed microbe will affect immunity or treatment against multiple cancers. For example, DNA fragments and/or genes encoding neoantigens and/or therapeutic targets of all lung cancers can be transfected into a microbe such that the microbe will affect immunity against all known lung cancers. Multiple recombinant plasmids or vectors could be used or chimeric methods could be used with a single recombinant plasmid or vector.

In a preferred embodiment, the exogenous DNA or gene is a DNA fragment or gene encoding a cancer neoantigen or other therapeutic target selectively expressed by cancer cells plus a DNA fragment or gene of a pathogen. To develop immunity against, or treat, a particular cancer, a DNA fragment or gene encoding a neoantigen or a therapeutic target that is selectively expressed by the cancer, plus a DNA fragment or gene of a pathogen, is transformed into a microbe in such a manner that the microbe expresses said DNA fragments or genes. The microbe is amplified by culturing. The transformed microbe is introduced to one or more of the mucous membranes, e.g., airways, GI tract, vagina. It can also be introduced the transdermally.

In a preferred embodiment, the exogenous DNA or gene encoding neoantigens, or other therapeutic targets, plus DNA fragment or gene of a pathogen, of multiple (more than one) types of cancer are transformed into a microbe such that the transformed microbe will affect immunity or treatment against multiple cancers. For example, DNA fragments and/or genes encoding neoantigens and/or therapeutic targets of all lung cancers can be transfected into a microbe such that the microbe will affect immunity against all known lung cancers. Multiple recombinant plasmids or vectors could be used or chimeric methods could be used with a single recombinant plasmid or vector. The addition of a DNA fragment or gene encoding a pathogen peptide, such as a piece of an endemic virus, is intended to trick the immune system to not recognize cancer neoantigens as ‘self’.

In a preferred embodiment, the exogenous DNA or gene is a DNA fragment or gene encoding a human peptide that is the target of autoimmunity. The rationale is to trick the immune system in extending tolerance to its target of autoimmunity by introducing a transformed microbe, especially one for which immune tolerance has been established, e.g., in the GI tract. Alternatively, transformed plant cells can be used to reorient the immune system to ‘see’ its autoimmunity target as ‘self’ antigens ingested in food. The concept is the same, except that plant cells are swapped for microbes.

In a preferred embodiment, the exogenous DNA or gene is a DNA fragment or gene encoding a peptide or protein of therapeutic value, such as insulin, ghrelin, elastin or collagen. To treat a particular disease, obesity, or reverse the appearance of aging, a DNA fragment or gene encoding a peptide or protein of known therapeutic value is transformed into a microbe in such a manner that the microbe expresses and secretes said DNA fragment or gene. The microbe is amplified by culturing. The transformed microbe is introduced to one or more of the mucous membranes, e.g., airways, GI tract, vagina. It can also be introduced the transdermally or on the skin.

In a preferred embodiment, the exogenous DNA or genes encoding multiple therapeutic targets (more than one) are transformed into a microbe such that the transformed microbe will affect a balanced treatment or more effective treatment. For example, DNA fragments and/or genes encoding elastin and collagen would be more effective at reversing the appearance of aging than either alone. Multiple recombinant plasmids or vectors could be used or chimeric methods could be used with a single recombinant plasmid or vector.

In a preferred embodiment, the exogenous DNA or gene is a DNA fragment or gene encoding a peptide or protein that is a known allergen. To prevent or treat an allergy or asthma, a DNA fragment or gene encoding a known allergen, or trigger, is transformed into a microbe in such a manner that the microbe expresses said DNA fragment or gene. The microbe is amplified by culturing. The transformed microbe is introduced to one or more of the mucous membranes, e.g., airways, GI tract, vagina. It can also be introduced the transdermally or on the skin.

In a preferred embodiment, the exogenous DNA or genes encoding multiple allergens (more than one) are transformed into a microbe such that the transformed microbe will affect a broad prevention against allergic reactions. For example, DNA fragments and/or genes encoding the most common allergens and triggers in children as a single treatment. Multiple recombinant plasmids or vectors could be used or chimeric methods could be used with a single recombinant plasmid or vector.

In one embodiment, it is an objective of this invention to present a method to prevent infectious diseases, in humans and animals, by transforming microbes, typically bacteria and fungi (but even possibly protozoa and plant cells), with genes encoding antigens of one or more infectious disease agent in a manner that causes the transformed microbe to express the antigens. The transformed microbe is then introduced to a body surface(s), including the skin, vagina, GI tract, and airways. The transformed microbe may also be applied transdermally. Unlike current methods of preventing infectious disease that involve the introduction of an antigen or a few antigens (vaccination), typically by injection, this invention relies on microbes to synthesize and present antigens in a natural, safer and more effective manner. Unlike vaccination, entire peptides comprising thousands of pathogen antigens are synthesized by the transformed microbe. These peptides, their chimera, or portions of the peptides are expressed by the transformed microbe, on its surface, in a manner that is ideal to be presented to the immune system. Because these peptides assume tertiary structures that are similar to those of the native peptides expressed by the target pathogen(s), the antigens are significantly more effective for generating an immune response—compared to the antigens used for vaccination. That combined with the fact that they are potentially thousands more antigens presented by transformed microbes, makes it a no-brainer why the transformed microbes would generate a more robust immune response and lasting protection from disease. The current status quo of countless trial and error to get effective vaccines and the need of booster shots is frustrating. Also, it takes too long to develop vaccines. Transformed microbes can be developed in a matter of days—compared to the several years it takes to create vaccines. People are also increasingly resisting vaccines. Frankly, vaccination is an antiquated practice that we have been holding unto because there was nothing better. This invention presents a significantly better option. Another major advantage of the transformed microbe antigen-presenting system is that it can be given to persons that are already ill from the target pathogen. In other words, it can be used for treatment as well as prevention.

In a preferred embodiment, peptides of target pathogens that are not themselves membrane proteins shall be made to insert into the cell membrane of transformed microbes. This is necessary, for example, to present viral peptides to the mucosal immune system. To achieve this, a signal sequence shall be attached to the coding sequence of the viral peptide. Also, transmembrane sequences shall be inserted into the coding sequence of the viral peptide. The result shall be to convert the peptide into a transmembrane peptide.

In another embodiment, it is an objective of this invention to present a method to treat infectious diseases, in humans and animals, by transforming microbes, typically bacteria and fungi (but even possibly protozoa and plant cells), with genes encoding antigens of one or more infectious disease agent in a manner that causes the transformed microbe to express the antigens. The transformed microbe is then distributed over a body surface(s), including the skin, vagina, GI tract, and airways. The transformed microbe may also be applied transdermally. Drug discovery and development takes several years and an enormous expenditure. And, although generally effective for their indications, drugs typically come with a list of adverse effects—some worse than the conditions the drugs are intended to treat. Additionally, infectious disease agents are generally good at mutating to render drugs ineffective over time. The immune system has an amazing capacity to defend and heal the body if antigens are presented to it in a timely and proper manner. The proper manner to present antigens to the immune system is via antigens in the various mucosal surfaces. Especially relevant, for the treatment of most diseases, is the GI tract mucosa which has the largest surface area of all the mucosae. Antigen presenting is more effective when it is done by a microbe because the immune system is obligated to deal with any and all microbes that find themselves on any mucosal surface. The presenting transformed microbe can be made to appear more menacing by employing a few tricks such as co-expressing the target antigens with antigens of another pathogen(s) especially one that is endemic to the region in which the treatment is being administered.

In another embodiment, it is an objective of this invention to present a method to prevent non-infectious diseases, such as cancers, in humans and animals, by transforming microbes, typically bacteria and fungi (but even possibly protozoa and plant cells), with genes encoding unique antigens (neoantigens) of one or more disease in a manner that causes the transformed microbe to express the antigens. The transformed microbe is then introduced to a body surface(s), including the skin, vagina, GI tract, and airways. The transformed microbe may also be applied transdermally. Unlike current methods of preventing disease that involve the introduction of an antigen or a few antigens (vaccination), typically by injection, this invention relies on microbes to synthesize and present antigens (neoantigens) in a natural, safer and more effective manner. Unlike vaccination, entire peptides comprising thousands neoantigens are synthesized by the transformed microbe. Multiple peptides from the same pathogen can be transformed into a microbe simultaneously. Multiple peptides from multiple pathogens can be transformed into a microbe simultaneously. These peptides, their chimera, or portions of the peptides are expressed by the transformed microbe, on its surface, in a manner that is ideal to be presented to the immune system. Because these peptides assume tertiary structures that are similar to those of the native peptides expressed by the disease, the antigens are significantly more effective for generating an immune response—compared to the antigens used for vaccination. That combined with the fact that they are potentially thousands more antigens presented by transformed microbes, makes it a no-brainer why the transformed microbes would generate a more robust immune response and lasting protection from disease. The current status quo of countless trial and error to get effective vaccines and the need of booster shots is frustrating. Also, it takes too long to develop vaccines. Transformed microbes can be developed in a matter of days—compared to the several years it takes to create vaccines.

In another embodiment, it is an objective of this invention to present a method to treat non-infectious diseases, such as cancer, in humans and animals, by transforming microbes, typically bacteria and fungi (but even possibly protozoa and plant cells), with genes encoding unique antigens (neoantigens) of one or more disease in a manner that causes the transformed microbe to express the antigens. The transformed microbe is then distributed over a body surface(s), including the skin, vagina, GI tract, and airways. The transformed microbe may also be applied transdermally. Drug discovery and development takes several years and an enormous expenditure. And, although generally effective for their indications, drugs typically come with a list of adverse effects—some worse than the conditions the drugs are intended to treat. Additionally, diseases such as cancer and Alzheimer's disease are still a long way away from reasonably effective drug therapy. The immune system has an amazing capacity to defend and heal the body if antigens are presented to it in a timely and proper manner. The proper manner to present antigens to the immune system is via antigens in the various mucosal surfaces. Especially relevant, for the treatment of most diseases, is the GI tract mucosa which has the largest surface area of all the mucosae. Antigen presenting is more effective when it is done by microbes because the immune system is obligated to deal with any and all microbes that find themselves on any mucosal surface. The presenting transformed microbe can be made to appear more menacing by employing a few tricks such as co-expressing the target antigens (neoantigens) with antigens of another pathogen(s) especially one that is endemic to the region in which the treatment is being administered. The advantages of this invention are numerous and colossal. For example, we can finally effectively treat cancer by region lung cancers, GI cancers, brain cancers, etc., by expressing the neoantigens of all cancers of a region in a microbe in a manner such that the transformed microbe is effective for preventing and treating all cancers of that region. This invention, in such regards, greatly simplifies the diagnosis, treatment, and prevention of disease.

In a preferred embodiment, peptides of target peptides that are not themselves membrane proteins shall be made to insert into the cell membrane of transformed microbes. This is necessary, for example, to present neoantigen peptides to the mucosal immune system. To achieve this, a signal sequence shall be attached to the coding sequence of the target peptide. Also, transmembrane sequences shall be inserted into the coding sequence of the target peptide. The result shall be to convert the peptide into a transmembrane peptide.

In another embodiment, it is an objective of this invention to present a method to prevent non-infectious diseases, such as allergies and asthma, in humans and animals, by transforming microbes, typically bacteria and fungi (but even possibly protozoa and plant cells), with genes encoding unique antigens (triggers) of one or more disease in a manner that causes the transformed microbe to express the antigens. The transformed microbe is then introduced to a body surface(s), including the skin, vagina, GI tract, and airways. The transformed microbe may also be applied transdermally. Unlike current methods of preventing disease that involve the introduction of an antigen or a few antigens (vaccination), typically by injection, this invention relies on microbes to synthesize and present antigens (triggers) in a natural, safer and more effective manner. Unlike vaccination, entire peptides comprising thousands of triggers are synthesized by the transformed microbe. These peptides, their chimera, or portions of the peptides are expressed by the transformed microbe, on its surface, in a manner that is ideal to be presented to the immune system. Because these peptides assume tertiary structures that are similar to those of the native disease triggers, the antigens are significantly more effective for generating an immune response—compared to the antigens used for vaccination. That combined with the fact that they are potentially thousands more antigens presented by transformed microbes, makes it a no-brainer why the transformed microbes would generate a more robust immune response and lasting protection from disease.

In a preferred embodiment, peptides of target triggers that are not themselves membrane proteins shall be made to insert into the cell membrane of transformed microbes. This is necessary, for example, to present trigger peptides to the mucosal immune system. To achieve this, a signal sequence shall be attached to the coding sequence of the trigger peptide. Also, transmembrane sequences shall be inserted into the coding sequence of the trigger peptide. The result shall be to convert the peptide into a transmembrane peptide.

In another embodiment, it is an objective of this invention to present a method to treat non-infectious diseases, such as allergies and asthma, in humans and animals, by transforming microbes, typically bacteria and fungi (but even possibly protozoa and plant cells), with genes encoding antigens of one or more disease triggers in a manner that causes the transformed microbe to express the antigens. The transformed microbe is then distributed over a body surface(s), including the skin, vagina, GI tract, and airways. The transformed microbe may also be applied transdermally. Drug discovery and development takes several years and an enormous expenditure. And, although generally effective for their indications, drugs typically come with a list of adverse effects—some worse than the conditions the drugs are intended to treat. The immune system has an amazing capacity to defend and heal the body if antigens are presented to it in a timely and proper manner. It can also be tricked to recognize a presented antigen as ‘self’ and hence to no longer attack the antigen. The proper manner to present antigens to the immune system is via antigens in the various mucosal surfaces. Especially relevant, for the treatment of most diseases, is the GI tract mucosa which has the largest surface area of all the mucosae. Antigen presenting is more effective when it is done by a microbe because the immune system is obligated to deal with any and all microbes that find themselves on any mucosal surface. Antigen presentation by plant cells, especially expressed in familiar foods, is an effective means of tricking the immune system to recognize the target (trigger) antigens as ‘self’.

In another embodiment, it is an objective of this invention to present a method to treat autoimmune diseases, in humans and animals, by transforming microbes, typically bacteria and fungi (but even possibly protozoa and plant cells), with genes encoding antigens of one or more disease triggers in a manner that causes the transformed microbe to express the antigens. The transformed microbe is then distributed over a body surface(s), including the skin, vagina, GI tract, and airways. The transformed microbe may also be applied transdermally. Drug discovery and development takes several years and an enormous expenditure. And, although generally effective for their indications, drugs typically come with a list of adverse effects—some worse than the conditions the drugs are intended to treat. The immune system has an amazing capacity to defend and heal the body if antigens are presented to it in a timely and proper manner. It can also be tricked to recognize a presented antigen as ‘self’ and hence to no longer attack the antigen. The proper manner to present antigens to the immune system is via antigens in the various mucosal surfaces. Especially relevant, for the treatment of most diseases, is the GI tract mucosa which has the largest surface area of all the mucosae. Antigen presenting is more effective when it is done by a microbe because the immune system is obligated to deal with any and all microbes that find themselves on any mucosal surface. Antigen presentation by plant cells, especially expressed in familiar foods, is an effective means of tricking the immune system to recognize the target (trigger) antigens as ‘self’.

In a preferred embodiment, peptides of target triggers that are not themselves membrane proteins shall be made to insert into the cell membrane of transformed microbes. This is necessary, for example, to present trigger peptides to the mucosal immune system. To achieve this, a signal sequence shall be attached to the coding sequence of the trigger peptide. Also, transmembrane sequences shall be inserted into the coding sequence of the trigger peptide. The result shall be to convert the peptide into a transmembrane peptide.

In another embodiment, it is an objective of this invention to present a method to prevent metabolic diseases and obesity, in humans and animals, by transforming microbes, typically bacteria and fungi (but even possibly protozoa and plant cells), with genes encoding peptides of one or more therapeutic targets in a manner that causes the transformed microbe to express the therapeutic target. The transformed microbe is then distributed to pertinent mucosae where expression of the therapeutic peptide will affect the desired therapeutic effect. For example, microbes can be transformed with genes encoding peptides such as cholecystokinin (CCK), glucagon-like peptide-1 (GLP-1), and peptide YY (PYY) and used like probiotics to prevent obesity or to prevent metabolic syndrome by reversing obesity.

In another embodiment, it is an objective of this invention to present a method to treat metabolic diseases and obesity, in humans and animals, by transforming microbes, typically bacteria and fungi (but even possibly protozoa and plant cells), with genes encoding peptides of one or more therapeutic targets in a manner that causes the transformed microbe to express the therapeutic target. The transformed microbe is then distributed to pertinent mucosae where expression of the therapeutic peptide will affect the desired therapeutic effect. For example, microbes can be transformed with genes encoding peptides such as cholecystokinin (CCK), glucagon-like peptide-1 (GLP-1), peptide YY (PYY) and insulin to actively treat and reverse obesity and metabolic syndrome.

In a preferred embodiment, peptides of target triggers that are not themselves membrane proteins (or secreted peptides) shall be made to insert into the cell membrane of transformed microbes or to be secreted by the microbe. This is necessary, for example, to present target peptides to the therapeutic targets. To achieve this, a signal sequence shall be attached to the coding sequence of the trigger peptide. That may suffice to secret the peptide. Also, transmembrane sequences shall be inserted into the coding sequence of the trigger peptide. The result shall be to convert the peptide into a transmembrane peptide or secreted peptide.

In one embodiment, it is an objective of this invention to present a method to rejuvenate and improve the appearance of skin, and minimize or reverse the appearance of aging, in humans and animals, by transforming microbes, typically bacteria and fungi (but even possibly protozoa and plant cells), with genes encoding pertinent peptides in a manner that causes the transformed microbe to express the peptides. The transformed microbe is then distributed over a body surface(s), including the skin, vagina, GI tract, and airways. The transformed microbe may also be applied transdermally. For example, microbes can be transformed with the genes encoding peptides such as collagen and elastin and said transformed microbes applied to the skin to rejuvenate it from outside in.

In a preferred embodiment, peptides of target triggers that are not themselves membrane proteins (or secreted peptides) shall be made to insert into the cell membrane of transformed microbes or to be secreted by the microbe. This is necessary, for example, to present target peptides to the therapeutic targets. To achieve this, a signal sequence shall be attached to the coding sequence of the trigger peptide. That may suffice to secret the peptide. Also, transmembrane sequences shall be inserted into the coding sequence of the trigger peptide. The result shall be to convert the peptide into a transmembrane peptide or secreted peptide.

It is an objective of this invention to present a method to diagnose diseases, in humans and animals, by transforming microbes, typically bacteria and fungi (but even possibly protozoa and plant cells), with genes encoding probing peptide(s) that interact with known disease markers (or products thereof) in a manner that causes the transformed microbe to express the probing peptide(s). The transformed microbe is then distributed over a body surface(s), including the skin, vagina, GI tract, and airways. The transformed microbe may also be applied transdermally.

In a preferred embodiment, the transformed microbes are presented in a pill formulation, liquid, or other regular pharmaceutical formulation at therapeutic doses.

In another embodiment, the transformed microbes are presented in foods and drinks, e.g., yoghurt and beer.

In another embodiment, the transformed microbes are presented as dried powder (spores and other quiescent states) that can be cultured and expanded.

Any formulation that can be ingested, inhaled, sprayed, applied, or inserted is appropriate for use with this technology.

### CITATIONS

The following cited works are hereby incorporated by reference in their entirety.
[1] R. Bingula, M. Filaire, N. Radosevic-Robin, et al., “Desired turbulence? Lung-gut axis, immunity and lung cancer,” Journal of Oncology, vol. 2017; 2017:5035371.
[2] A. M. Brownawell, W. Caers, G. R. Gibson et al., “Prebiotics and the health benefits of fiber: Current regulatory status, future research, and goals,” Journal of Nutrition, vol. 142, no. 5, pp. 962-974, 2012.
[3] Y. Belkaid and T. W. Hand, “Role of the microbiota in immunity and inflammation,” Cell, vol. 157, no. 1, pp. 121-141, 2014.
[4] S. Carding, K. Verbeke, D. T. Vipond, B. M. Corfe, and L. J. Owen, “Dysbiosis of the gut microbiota in disease,” Microbial Ecology in Health & Disease, vol. 26, no. 1, 2015.
[5] P. Forsythe, W. Kunze, and J. Bienenstock, “Moody microbes or fecal phrenology: What do we know about the microbiota gut-brain axis?” BMC Medicine, vol. 14, no. 1, article no. 58, 2016.
[6] T. J. Schuijt, J. M. Lankelma, B. P. Scicluna et al., “The gut microbiota plays a protective role in the host defence against pneumococcal pneumonia,” Gut, vol. 65, no. 4, pp. 575-583, 2016.
[7] M. Hilty, C. Burke, H. Pedro et al., “Disordered microbial communities in asthmatic airways,” PLoS ONE, vol. 5, no. 1, Article ID e8578, 2010.
[8] H. Wang, J. S. Liu, S. H. Peng et al., “Gut-lung crosstalk in pulmonary involvement with inflammatory bowel diseases,” World Journal of Gastroenterology, vol. 19, no. 40, pp.-94-6804, 2013.
[9] T. Jess, E. Horvath-Puho, J. Fallingborg, H. H. Rasmussen, and B. A. Jacobsen, “Cancer risk in inflammatory bowel disease according to patient phenotype and treatment: a danish population-based cohort study,” American Journal of Gastroenterology, vol. 108, no. 12, pp. 1869-1876, 2013.
[10] S. Keely, N. J. Talley, and P. M. Hansbro, “Pulmonary-intestinal cross-talk in mucosal inflammatory disease,” Mucosal Immunology, vol. 5, no. 1, pp. 7-18, 2012.
[11] J. Tap, S. Mondot, F. Levenez et al., “Towards the human intestinal microbiota phylogenetic core,” Environmental Microbiology, vol. 11, no. 10, pp. 2574-2584, 2009.
[12] V. Lazarevic, K. Whiteson, S. Huse et al., “Metagenomic study of the oral microbiota by Illumina high-throughput sequencing,” Journal of Microbiological Methods, vol. 79, no. 3, pp. 266-271, 2009.
[13] K. P. Lemon, V. Klepac-Ceraj, H. K. Schiffer, E. L. Brodie, S. V. Lynch, and R. Kolter, “Comparative analyses of the Bacterial microbiota of the human nostril and oropharynx,” mBio, vol. 1, no. 3, 2010.
[14] E. S. Charlson, K. Bittinger, A. R. Haas et al., “Topographical continuity of bacterial populations in the healthy human respiratory tract,” The American Journal of Respiratory and Critical Care Medicine, vol. 184, no. 8, pp. 957-963, 2011.
[15] Z. Pei, E. J. Bini, L. Yang, M. Zhou, F. Francois, and M. J. Blaser, “Bacterial biota in the human distal esophagus,” Proceedings of the National Academy of Sciences of the United States of America, vol. 101, no. 12, pp. 4250-4255, 2004.
[16] A. A. Pragman, H. B. Kim, C. S. Reilly, C. Wendt, and R. E. Isaacson, “The Lung Microbiome in Moderate and Severe Chronic Obstructive Pulmonary Disease,” PLoS ONE, vol. 7, no. 10, Article ID e47305, 2012.
[17] J. R. Erb-Downward, D. L. Thompson, M. K. Han et al., “Analysis of the lung microbiome in the ‘healthy’ smoker and in COPD,” PLoS ONE, vol. 6, no. 2, Article ID e16384, 2011.
[18] M. Garcia-Nunez, L. Millares, X. Pomares et al., “Severity related changes of bronchial microbiome in chronic obstructive pulmonary disease,” Journal of Clinical Microbiology, vol. 52, no. 12, pp. 4217-4223, 2014.
[19] M. A. Sze, P. A. Dimitriu, S. Hayashi et al., “The lung tissue microbiome in chronic obstructive pulmonary disease,” American Journal of Respiratory and Critical Care Medicine, vol. 185, no. 10, pp. 1073-1080, 2012.
[20] D. Wu, C. Hou, Y. Li et al., “Analysis of the bacterial community in chronic obstructive pulmonary disease sputum samples by denaturing gradient gel electrophoresis and real-time PCR,” BMC Pulmonary Medicine, vol. 14, no. 1, article no. 179, 2014.
[21] X. Yan, M. Yang, J. Liu et al., “Discovery and validation of potential bacterial biomarkers for lung cancer,” American Journal of Cancer Research, vol. 5, pp. 3111-3122, 2015.
[22] P. Rybojad, R. Los, M. Sawicki, J. Tabarkiewicz, and A. Malm, “Anaerobic bacteria colonizing the lower airways in lung cancer patients,” Folia Histochemica et Cytobiologica, vol. 49, no. 2, pp. 263-266, 2011.
[23] M. T. Abreu, “Toll-like receptor signaling in the intestinal epithelium: how bacterial recognition shapes intestinal function,” Nature Reviews Immunology, vol. 10, no. 2, pp. 131-144, 2010.
[24] G. D. Wu, J. Chen, C. Hoffmann et al., “Linking long-term dietary patterns with gut microbial enterotypes,” Science, vol. 334, no. 6052, pp. 105-108, 2011.
[25] M. Arumugam, J. Raes, E. Pelletier et al., “Enterotypes of the human gut microbiome,” Nature, vol. 12, pp. 174-180, 2011.
[26] D. R. Samuelson, D. A. Welsh, and J. E. Shellito, “Regulation of lung immunity and host defense by the intestinal microbiota,” Frontiers in Microbiology, vol. 6, article no. 1085, 2015.
[27] I. I. Ivanov and K. Honda, “Intestinal commensal microbes as immune modulators,” Cell Host& Microbe, vol. 12, no. 4, pp. 496-508, 2012.
[28] A. Hakansson and G. Molin, “Gut microbiota and inflammation,” Nutrients, vol. 3, no. 6, pp. 637-682, 2011.
[29] B. Kelsall, “Recent progress in understanding the phenotype and function of intestinal dendritic cells and macrophages,” Mucosal Immunology, vol. 1, no. 6, pp. 460-469, 2008.
[30] T. Nakajima, V. Palchevsky, D. L. Perkins, J. A. Belperio, and P. W. Finn, “Lung transplantation: Infection, inflammation, and the microbiome,” Seminars in Immunopathology, vol. 33, no. 2, pp. 135-156, 2011.
[31] A. J. Macpherson and T. Uhr, “Induction of protective IgA by intestinal dendritic cells carrying commensal bacteria. Science,” Science, vol. 303, no. 5664, pp. 1662-1665, 2004.
[32] H. Tezuka, Y. Abe, J. Asano et al., “Prominent Role for Plasmacytoid Dendritic Cells in Mucosal T Cell-Independent IgA Induction,” Immunity, vol. 34, no. 2, pp. 247-257, 2011.
[33] E. Castigli, S. A. Wilson, S. Scott et al., “TACI and BAFF-R mediate isotype switching in B cells,” Journal of Experimental Medicine, vol. 201, no. 1, pp. 35-39, 2005.
[34] N. Kamada, S. U. Seo, G. Y. Chen, and G. Nunez, “Role of the gut microbiota in immunity and inflammatory disease,” Nature Reviews Immunology, vol. 13, no. 5, pp. 321-335, 2013.
[35] P. Lopez, M. Gueimonde, A. Margolles, and A. Suarez, “Distinct Bifidobacterium strains drive different immune responses in vitro,” International Journal of Food Microbiology, vol. 138, no. 1-2, pp. 157-165, 2010.
[36] H. S. Gill, K. J. Rutherfurd, M. L. Cross, and P. K. Gopal, “Enhancement of immunity in the elderly by dietary supplementation with the probiotic Bifidobacterum lactis HN019,” American Journal of Clinical Nutrition, vol. 74, no. 6, pp. 833-839, 2001.
[37] G. T. Macfarlane and S. Macfarlane, “Bacteria, colonic fermentation, and gastrointestinal health,” Journal of AOAC International, vol. 95, no. 1, pp. 50-60, 2012.
[38] D. L. Topping and P. M. Clifton, “Short-chain fatty acids and human colonic function: roles of resistant starch and non-starch polysaccharides,” Physiological Reviews, vol. 81, no. 3, pp. 1031-1064, 2001.
[39] G. T. Macfarlane and S. Macfarlane, “Fermentation in the human large intestine: Its physiologic consequences and the Journal of Oncology 13 potential contribution of prebiotics,” Journal of Clinical Gastroenterology, vol. 45, no. 3, pp. S120-S127, 2011.
[40] E. le Poul, C. Loison, S. Struyf et al., “Functional characterization of human receptors for short chain fatty acids and their role in polymorphonuclear cell activation,” The Journal of Biological Chemistry, vol. 278, no. 28, pp. 25481-25489, 2003.
[41] N. Singh, A. Gurav, S. Sivaprakasam et al., “Activation of Gpr109a, receptor for niacin and the commensal metabolite butyrate, suppresses colonic inflammation and carcinogenesis,” Immunity, vol. 40, no. 1, pp. 128-139, 2014.
[42] N. I. McNeil, “The contribution of the large intestine to energy supplies in man,” The American Journal of Clinical Nutrition, vol. 39, no. 3, pp. 338-342, 1981.
[43] S. Krishnan, N. Alden, and K. Lee, “Pathways and functions of gut microbiota metabolism impacting host physiology,” Current Opinion in Biotechnology, vol. 36, pp. 137-145, 2015.
[44] Y. Furusawa, Y. Obata, S. Fukuda et al., “Commensal microbe-derived butyrate induces the differentiation of colonic regulatory T cells,” Nature, vol. 504, no. 7480, pp. 446-450, 2013.
[45] L. Chen, W. Fischle, E. Verdin, and W. C. Greene, “Duration of nuclear NF-κB action regulated by reversible acetylation,” Science, vol. 293, no. 5535, pp. 1653-1657, 2001.
[46] Z.-L. Yuan, Y.-J. Guan, D. Chatterjee, and Y. E. Chin, “Stat3 dimerization regulated by reversible acetylation of a single lysine residue,” Science, vol. 307, no. 5707, pp. 269-273, 2005.
[47] S. Viaud, R. Daillere, I. G. Boneca et al., “Gut microbiome and anticancer immune response: really hot Sh*t!,” Cell Death and Differentiation, 2014.
[48] H. M. Hamer, D. Jonkers, K. Venema, S. Vanhoutvin, F. J. Troost, and R. J. Brummer, “Review article: the role of butyrate on colonic function,” Alimentary Pharmacology and Therapeutics, vol. 27, no. 2, pp. 104-119, 2008.
[49] E. Cario, “Microbiota and innate immunity in intestinal inflammation and neoplasia,” Current Opinion in Gastroenterology, vol. 29, no. 1, pp. 85-91, 2013.
[50] S. Dulal and T. O. Keku, “Gut microbiome and colorectal adenomas,” Cancer Journal (United States), vol. 20, no. 3, pp. 225-231, 2014.
[51] L. V. Hooper, D. R. Littman, and A. J. Macpherson, “Interactions between the microbiota and the immune system,” Science, vol. 336, no. 6086, pp. 1268-1273, 2012.
[52] C. T. Fagundes, F. A. Amaral, A. T. Vieira et al., “Transient TLR activation restores inflammatory response and ability to control pulmonary bacterial infection in germfree mice,” The Journal of Immunology, vol. 188, no. 3, pp. 1411-1420, 2012.
[53] I. C. Allen, E. M. Tekippe, R. M. T. Woodford et al., “TheNLRP3 inflammasome functions as a negative regulator of tumorigenesis during colitis-associated cancer,” Journal of Experimental Medicine, vol. 207, no. 5, pp. 1045-1056, 2010.
[54] D. S. Chen and I. Mellman, “Oncology meets immunology: the cancer-immunity cycle,” Immunity, vol. 39, no. 1, pp. 1-10, 2013.
[55] T. W. Hand, L. M. Dos Santos, N. Bouladoux et al., “Acute gastrointestinal infection induces long-lived microbiota-specific T cell responses,” Science, vol. 337, no. 6101, pp. 1553 1556, 2012.
[56] E. Slack, S. Hapfelmeier, B. Stecher et al., “Innate and adaptive immunity cooperate flexibly to maintain host-microbiota mutualism,” Science, vol. 325, no. 5940, pp. 617-620, 2009.
[57] N. Iida, A. Dzutsev, C. A. Stewart et al., “Commensal bacteria control cancer response to therapy by modulating the tumor microenvironment,” Science, vol. 342, no. 6161, pp. 967-970, 2013.
[58] S. Viaud, F. Saccheri, G. Mignot et al., “The intestinal microbiota modulates the anticancer immune effects of cyclophosphamide,” Science, vol. 342, no. 6161, pp. 971-976, 2013.
[59] L. Zitvogel, M. Ayyoub, B. Routy, and G. Kroemer, “Microbiome and Anticancer Immunosurveillance,” Cell, vol. 165, no. 2, pp. 276-287, 2016.
[60] Q. F. Gui, H. F. Lu, C. X. Zhang, Z. R. Xu, Y. H. Yang, “Well-balanced commensal microbiota contributes to anti-cancer response in a lung cancer mouse model,” Genet Mol Res. Vol. 14, no. 2, pp. 5642-51, 2015.
[61] K. L. Flannigan, D. Geem, A. Harusato, and T. L. Denning, “Intestinal Antigen-Presenting Cells Key Regulators of Immune Homeostasis and Inflammation,” The American Journal of Pathology, Vol. 185, no. 7, 2015.
[62] K. J. Maloy, F. Powrie, “Intestinal homeostasis and its breakdown in inflammatory bowel disease,” Nature, vol. 474, pp. 298-306, 2011.
[63] A. M. Mowat, W. W, Agace, “Regional specialization within the intestinal immune system,” Nat Rev Immunol, vol. 14, pp. 667-685, 2014.
[64] C. C. Bain, A. M. Mowat, “Macrophages in intestinal homeostasis and inflammation,” Immunol Rev, vol. 260, pp. 102-117, 2014.
[65] C. C. Bain, A. M. Mowat, “Intestinal macrophages: specialised adaptation to a unique environment,” Eur J Immunol, vol. 41, pp. 2494-2498, 2011.
[66] J. L. Coombes, F. Powrie, “Dendritic cells in intestinal immune regulation,” Nat Rev Immunol, vol. 8, pp. 435-446, 2008.
[67] A. M. Mowat, C. C. Bain, “Mucosal macrophages in intestinal homeostasis and inflammation, J Innate Immun, vol. 3, pp. 550-564, 2011.
[68] O. Pabst, G. Bernhardt, The puzzle of intestinal lamina propria dendritic cells and macrophages, Eur J Immunol, vol. 40, pp. 2107-2111, 2010.
[69] P.D. Smith, L. E. Smythies, R. Shen, T. Greenwell-Wild, M. Gliozzi, S. M. Wahl, Intestinal macrophages and response to microbial encroachment, Mucosal Immunol, vol. 4, pp. 31-42, 2011.
[70] P. D. Smith, C. Ochsenbauer-Jambor, L. E. Smythies, “Intestinal macrophages: unique effector cells of the innate immune system,” Immunol Rev, vol. 206, pp. 149-159, 2005.
[71] C. Abraham, R. Medzhitov, Interactions between the host innate immune system and microbes in inflammatory bowel disease, Gastroenterology, vol. 140, pp. 1729-1737, 2011.
[72] C. C. Bain, A. Bravo-Blas, C. L. Scott, E. Gomez Perdiguero, F. Geissmann, S. Henri, B. Malissen, L. C. Osborne, D. Artis, A. M. Mowat, “Constant replenishment from circulating monocytes maintains the macrophage pool in the intestine of adult mice,” Nat Immunol, vol. 15, pp. 929-937, 2014.
[73] C. C. Bain, C. L. Scott, H. Uronen-Hansson, S. Gudjonsson, O. Jansson, O. Grip, M. Guilliams, B. Malissen, W. W. Agace, A. M. Mowat, “Resident and pro-inflammatory macrophages in the colon represent alternative context-dependent fates of the same Ly6Chi monocyte precursors,” Mucosal Immunol, vol. 6, pp. 498-510, 2013.
[74] S. Tamoutounour, S. Henri, H. Lelouard, B. de Bovis, C. de Haar, C. J. van der Woude, A. M. Woltman, Y. Reyal, D. Bonnet, D. Sichien, C. C. Bain, A. M. Mowat, C. Reis e Sousa, L. F. Poulin, B. Malissen, M. Guilliams, “CD64 distinguishes macrophages from dendritic cells in the gut and reveals the Th1-inducing role of mesenteric lymph node macrophages during colitis,” Eur J Immunol, vol. 42, pp. 3150-3166, 2012.
[75] E. Zigmond, C. Varol, J. Farache, E. Elmaliah, A. T. Satpathy, G. Friedlander, M. Mack, N. Shpigel, I. G. Boneca, K. M. Murphy, G. Shakhar, Z. Halpern, S. Jung, “Ly6C hi monocytes in the inflamed colon give rise to proinflammatory effector cells and migratory antigen-presenting cells,” Immunity, vol. 37, pp. 1076-1090, 2012.
[76] D. K. Fogg, C. Sibon, C. Miled, S. Jung, P. Aucouturier, D. R. Littman, A. Cumano, F. Geissmann, “A clonogenic bone marrow progenitor specific for macrophages and dendritic cells,” Science, vol. 311, pp. 83-87, 2006.
[77] M. Bogunovic, F. Ginhoux, J. Helft, L. Shang, D. Hashimoto, M. Greter, K. Liu, C. Jakubzick, M. A. Ingersoll, M. Leboeuf, E. R. Stanley, M. Nussenzweig, S. A. Lira, G. J. Randolph, M. Merad, “Origin of the lamina propria dendritic cell network,” Immunity, vol. 31, pp. 513-525, 2009.
[78] K. P. MacDonald, J. S. Palmer, S. Cronau, E. Seppanen, S. Olver, N. C. Raffelt, R. Kuns, A. R. Pettit, A. Clouston, B. Wainwright, D. Branstetter, J. Smith, R. J. Paxton, D. P. Cerretti, L. Bonham, G. R. Hill, D. A. Hume, “An antibody against the colony-stimulating factor 1 receptor depletes the resident subset of monocytes and tissue-and tumor-associated macrophages but does not inhibit inflammation,” Blood, vol. 116, pp. 3955-3963, 2010.
[79] G. R. Ryan, X. M. Dai, M. G. Dominguez, W. Tong, F. Chuan, O. Chisholm, R. G. Russell, J. W. Pollard, E. R. Stanley, “Rescue of the colony stimulating factor 1 (CSF-1)-nullizygous mouse (Csf1(op)/Csf1(op)) phenotype with a CSF-1 transgene and identification of sites of local CSF-1 synthesis,” Blood, vol. 98, pp. 74-84, 2001.
[80] P. de Santa Barbara, G. R. van den Brink, D. J. Roberts, “Molecular etiology of gut malformations and diseases,” Am J Med Genet, vol. 115, pp. 221-30, 2002.
[81] T. T. MacDonald, G. Monteleone, “Immunity, inflammation & allergy in the gut” Science, vol. 307, pp. 1920-1925, 2005.
[82] L. V. Hooper, “Epithelial cell contributions to intestinal immunity,” Adv Immunol, vol. 126, pp. 129-172.
[83] J. R. McDole, L. W. Wheeler, K. G. McDonald, B. Wang, V. Konjufca, K. A. Knoop, et al., “Goblet cells deliver luminal antigen to CD103+ dendritic cells in the small intestine,” Nature, vol. 483, pp. 345-349, 2012.
[84] K. A. Knoop, K. G. McDonald, S. McCrate, J. R. McDole, R. D. Newberry, “Microbial sensing by goblet cells controls immune surveillance of luminal antigens in the colon,” Mucosal Immunol, vol. 8, pp. 198-210, 2015.
[85] O. Schulz, O. Pabst, “Antigen sampling in the small intestine,” Trends Immunol, vol. 34, pp. 2013.
[86] J. H. Niess, S. Brand, X. Gu, L. Landsman, S. Jung, B. A. McCormick, et al., “CX3CR1-mediated dendritic cell access to the intestinal lumen and bacterial clearance,” Science, vol. 307, pp. 254-258, 2005.
[87] S. Kerneis, A. Bogdanova, J. P. Kraehenbuhl, E. Pringault, “Conversion by Peyer's patch lymphocytes of human enterocytes into M cells that transport bacteria,” Science, vol 277, pp. 949-952.
[88] F. Gerbe, E. Sidot, D. J. Smyth, M. Ohmoto, I. Matsumoto, V. Dardalhon, et al., “Intestinal epithelial tuft cells initiate type 2 mucosal immunity to helminth parasites,” Nature, vol. 529, pp. 226-230, 2016.
[89] A. Banerjee, E. T. McKinley, J. von Moltke, R. J. Coffey, K. S. Lau, “Interpreting heterogeneity in intestinal tuft cell structure and function,” J Clin Invest, vol. 128, pp. 1711-1719, 2018.
[90] R. Okumura, K. Takeda, “Roles of intestinal epithelial cells in the maintenance of gut homeostasis,” Exp Mol Med, vol. 49, pp. 338, 2017.
[91] R. Okumura, K. Takeda, “Maintenance of intestinal homeostasis by mucosal barriers,” Inflamm Regen, vol. 38, pp. 5, 2018.
[92] M. Fukata, M. Arditi, “The role of pattern recognition receptors in intestinal inflammation. Mucosal Immunol, vol. 6, pp. 451-463.
[93] J. M. Otte, E. Cario, D. K. Podolsky, “Mechanisms of cross hyporesponsiveness to Toll-like receptor bacterial ligands in intestinal epithelial cells,” Gastroenterology, vol. 126, pp. 1054-1070, 2004.
[94] G. Melmed, L. S. Thomas, N. Lee, S. Y. Tesfay, K. Lukasek, K. S. Michelsen, et al., “Human intestinal epithelial cells are broadly unresponsive to Toll-like receptor 2-dependent bacterial ligands: implications for host microbial interactions in the gut,” J Immunol, vol. 170, pp. 1406-1415.
[95] P.P. Jones, D. B. Murphy, D. Hewgill, H. O. McDevitt, “Detection of a common polypeptide chain in IA and IE sub-region immunoprecipitates,” Mol Immunol, vol. 16, pp. 51-60, 1979.
[96] M. Cloutier, C. Gauthier, J. S. Fortin, L. Geneve, K. Kim, S. Gruenheid, et al., “ER egress of invariant chain isoform p35 requires direct binding to MHCII molecules and is inhibited by the NleA virulence factor of enterohaemorrhagic Escherichia coli,” Hum Immunol, vol. 76, pp. 292-296, 2015.
[97] J. Neefjes, M. L. M. Jongsma, P. Paul, O. Bakke, “Towards a systems understanding of MHC class I and MHC class II antigen presentation,” Nat Rev Immunol, vol. 11, pp. 823-836, 2011.
[98] E. D. Mellins, L. J. Stern, “HLA-DM and HLA-DO, key regulators of MHCII processing and presentation,” Curr Opin Immunol, vol. 26, pp. 115-122, 2014.
[99] A. I. Guce, S. E. Mortimer, T. Yoon, C. A. Painter, W. Jiang, E. D. Mellins, et al., “HLA-DO acts as a substrate mimic to inhibit HLA-DM by a competitive mechanism,” Nat Struct Mol Biol, vol. 20, pp. 90-98, 2013.
[100] R. J. Greenwald, G. J. Freeman, A. H. Sharpe, “The B7 family revisited. Annu Rev Immunol, vol. 23, pp. 515-548, 2005.
[101] H. Hemmi, O. Takeuchi, T, Kawai, T, Kaisho, S, Sato, H. Sanjo, et al., “A toll-like receptor recognizes bacterial DNA,” Nature, vol. 408, pp. 740-745, 2000.
[102] M. L. Alegre, K. A. Frauwirth, C. B. Thompson, “T-cell regulation by CD28 and CTLA-4. Nat Rev Immunol, vol. 1, pp. 220-228, 2001.
[103] G. Mayrhofer, C. W. Pugh, A. N. Barclay, “The distribution, ontogeny and origin in the rat of Ia-positive cells with dendritic morphology and of Ia antigen in epithelia, with special reference to the intestine,” Eur J Immunol, vol. 13, pp. 112-122, 1983.
[104] K. Wiman, B. Curman, U. Forsum, L. Klareskog, U. MalmnäS-Tjernlund, L. Rask, et al., “Occurrence of Ia antigens on tissues of non-lymphoid origin,” Nature, vol. 276, pp. 711-713, 1978.
[105] E. L. Parr, I. F. C. McKenzie, “Demonstration of Ia antigens on mouse intestinal epithelial cells by immunoferritin labeling,” Immunogenetics, vol. 8, pp. 499-508, 1979.
[106] M. Chiba, M. Iizuka, O. Masamune, “Ubiquitous expression of HLA-DR antigens on human small intestinal epithelium, Gastroenterol Jpn, vol. 23, pp. 109-116, 1988.
[107] X. P. Lin, N. Almqvist, E. Telemo, “Human small intestinal epithelial cells constitutively express the key elements for antigen processing and the production of exosomes,” Blood Cells Mol Dis, vol. 35, pp. 122-128, 2005.
[108] B. Byrne, L. Madrigal-Estebas, A. McEvoy, J. Carton, D. G. Doherty, A. Whelan, et al., “Human duodenal epithelial cells constitutively express molecular components of antigen presentation but not costimulatory molecules,” Hum Immunol, vol. 63, pp. 977-986.
[109] T. MacDonald, A. Weinel, J. Spencer, “HLA-DR expression in human fetal intestinal epithelium,” Gut, vol. 29, pp. 1342-1348, 1988.
[110] A. M. Oliver, A. W. Thomson, H. F. Sewell, D. R. Abramovch, “Major histocompatibility complex (MHC) class II antigen (HLA-DR, DQ & DP) expression in human fetal endocrine organs and gut,” Scand J Immunol, vol. 27, pp. 731-737, 1988.
[111] P. G. Natali, C. De Martino, M. A. Pellegrino, S. Ferrone, “Analysis of the expression of I-Ak-like antigens in murine fetal and adult tissues with the monoclonal antibody 10-2.16,” Scand J Immunol, vol. 13, pp. 541-546, 1981.
[112] H. Scott, B. G. Solheim, P. Brandtzaeg, E. Thorsby, “HLA-DR-like antigens in the epithelium of the human small intestine,” Scand J Immunol, vol. 12, pp. 77-82, 1980.
[113] K. Koretz, F. Momburg, H. F. Otto, P. Moller, “Sequential induction of MHC antigens on autochthonous cells of ileum affected by Crohn's disease,” Am J Pathol, vol. 129, pp. 493-502, 1987.
[114] F. Momburg, K. Koretz, A. Von Herbay, P. Moller, “Nonimmune human cells can express MHC class II antigens in the absence of invariant chain-an immunohistological study on normal and chronically inflamed small intestine,” Clin Exp Immunol, vol. 72, pp. 367-372, 1988.
[115] S. Fais, L. Maiuri, F. Pallone, M. De Vincenzi, G. De Ritis, R. Troncone, et al., “Gliadin induced changes in the expression of MHC-class II antigens by human small intestinal epithelium. Organ culture studies with coeliac disease mucosa,” Gut, vol. 33, pp. 472-475, 1992.
[116] J. Kelly, C. O'Farrelly, C. O'Mahony, D. Weir, C. Feighery, “Immunoperoxidase demonstration of the cellular composition of the normal and coeliac small bowel,” Clin Exp Immunol, vol. 68, pp. 177, 1987.
[117] D. W. Mason, M. Dallman, A. N. Barclay, “Graft-versus-host disease induces expression of Ia antigen in rat epidermal cells and gut epithelium, Nature, vol. 293, pp. 150-151, 1981.
[118] I. Dotan, M. Allez, A. Nakazawa, J. Brimnes, M. Schulder-Katz, L. Mayer, “Intestinal epithelial cells from inflammatory bowel disease patients preferentially stimulate CD4+ T cells to proliferate and secrete interferon-g, Am J Physiol. Gastrointest Liver Physiol, vol. 292, pp. 1630-1640, 2007.
[119] L. Mayer, D. Eisenhardt, P. Salomon, W. Bauer, R. Plous, L. Piccinini, “Expression of class II molecules on intestinal epithelial cells in humans. Differences between normal and inflammatory bowel disease,” Gastroenterology, vol. 100, pp. 3-12, 1991.
[120] K. P. Zimmer, C. Poremba, P. Weber, P. J. Ciclitira, E. Harms, “Translocation of gliadin into HLA-DR antigen containing lysosomes in coeliac disease enterocytes,” Gut, vol. 36, pp. 703-709, 1995.
[121] F. Arnaud-Battandier, N. Cerf-Bensussan, R. Amsellem, J. Schmitz, “Increased HLA-DR expression by enterocytes in children with celiac disease,” Gastroenterology, vol. 91, pp. 1206-1212, 1986.
[122] R. Rojas, G. Apodaca, “Immunoglobulin transport across polarized epithelial cells,” Nat Rev Mol Cell Biol, vol. 3, pp. 944-956, 2002.
[123] A. S. Daar, S. V. Fuggle, J. W. Fabre, A. Ting, P. J. Morris, “The detailed distribution of class II antigens in normal human organs,” Transplantation, vol. 38. pp. 293-298, 1984.
[124] I. Hirata, L. L. Austin, W. H. Blackwell, J. R. Weber, W. O. Dobbins III, “Immunoelectron microscopic localization of HLA-DR antigen in control small intestine and colon and in inflammatory bowel disease,” Digest Dis Sci, vol. 31, pp. 1317-1330, 1986.
[125] J. Sarles, J. P. Gorvel, D. Olive, S. Maroux, C. Mawas, F. Giraud, “Subcellular localization of class I (A, B, C) and class II (DR and DQ) MHC antigens in jejunal epithelium of children with coeliac disease,” J Pediatr Gastroenterol Nutr, vol. 6, pp. 51-56, 1987.
[126] G. Hundorfean, K. P. Zimmer, S. Strobel, A. Gebert, D. Ludwig, J. Büing, “Luminal antigens access late endosomes of intestinal epithelial cells enriched in MHC I and MHC II molecules: in vivo study in Crohn's ileitis,” Am J Physiol Gastrointest Liver Physiol, vol. 293, pp. 798-808, 2007.
[127] R. M. Hershberg, D. H. Cho, A. Youakim, M. B. Bradley, J. S. Lee, P. E. Framson, et al., “Highly polarized HLA class II antigen processing and presentation by human intestinal epithelial cells,” J Clin Invest, vol. 102, pp. 792-803, 1998.
[128] A. M. Westendorf, D. Fleissner, L. Groebe, S. Jung, A. D. Gruber, W. Hansen, et al., “CD4+Foxp3+regulatory T cell expansion induced by antigen-driven interaction with intestinal epithelial cells independent of local dendritic cells,” Gut, vol. 58, pp. 211-219, 2009.
[129] C. Thelemann, R. O. Eren, M. Coutaz, J. Brasseit, H. Bouzourene, M. Rosa, et al., “Interferon-g induces expression of MHC class II on intestinal epithelial cells and protects mice from colitis,” PLoS ONE 9: e86844, 2014.
[130] M. Uhlen, L. Fagerberg, B. M. Hallstrom, C. Lindskog, P. Oksvold, A. Mardinoglu, et al., “Tissue-based map of the human proteome,” Science, vol. 347, 2015.
[131] E. Walseng, K. Furuta, R. S. Goldszmid, K. A. Weih, A. Sher, P. A. Roche, “Dendritic cell activation prevents MHC class II ubiquitination and promotes MHC class II survival regardless of the activation stimulus, J Biol Chem, vol. 285, pp. 41749-54, 2010.
[132] M. A. Sze, M. Tsuruta, S.-W. J. Yang et al., “Changes in the bacterial microbiota in gut, blood, and lungs following acute LPS instillation into mice lungs,” PLoS ONE, vol. 9, no. 10, 2014.
[133] A. F. Goddard, B. J. Staudinger, S. E. Dowd et al., “Direct sampling of cystic fibrosis lungs indicates that DNA-based analyses of upper-airway specimens can misrepresent lung microbiota,” Proceedings of the National Academy of Sciences of the United States of America, vol. 109, no. 34, pp. 13769-13774, 2012.
[134] A. Venkataraman, C. M. Bassis, J. M. Beck et al., “Application of a neutral community model to assess structuring of the human lung microbiome,” mBio, vol. 6, no. 1, 2015.
[135] R. A. Stockley, “Measurement of soluble proteins in lung secretion,” Thorax, vol. 39, no. 4, pp. 241-247, 1984.
[136] I. Fillion, N. Ouellet, M. Simard, Y. Bergeron, S. Sato, and M. G. Bergeron, “Role of chemokines and formyl peptides in pneumococcal pneumonia-induced monocyte/macrophage recruitment,” The Journal of Immunology, vol. 166, no. 12, pp. 7353-7361, 2001.
[137] L.-F. Lee, R. P. Hellendall, Y. Wang et al., “IL-8 reduced tumorigenicity of human ovarian cancer in vivo due to neutrophil infiltration,” Journal of Immunology, vol. 164, no. 5, pp. 2769-2775, 2000.
[138] P. J. Barnes, “Chronic obstructive pulmonary disease,” The New England Journal of Medicine, vol. 343, no. 4, pp. 269-280, 2000.
[139] D. Worlitzsch, R. Tarran, M. Ulrich et al., “Effects of reduced mucus oxygen concentration in airway Pseudomonas infections of cystic fibrosis patients,” The Journal of Clinical Investigation, vol. 109, no. 3, pp. 317-325, 2002.
[140] A. Schmidt, A. Belaaouaj, R. Bissinger et al., “Neutrophil elastase-mediated increase in airway temperature during inflammation,” Journal of Cystic Fibrosis, vol. 13, no. 6, pp.-3-631, 2014.
[141] P. P. Freestone, R. A. Hirst, S. M. Sandrini et al., “Pseudomonas aeruginosa-catecholamine inotrope interactions: A contributory factor in the development of ventilator-associated pneumonia?” Chest, vol. 142, no. 5, pp. 1200-1210, 2012.
[142] W. Lin and M. Karin, “A cytokine-mediated link between innate immunity, inflammation, and cancer,” Journal of Clinical Investigation, vol. 117, no. 5, pp. 1175-1183, 2007.
[143] A. R. Davalos, J. P. Coppe, J. Campisi, and P. Y. Desprez, “Senescent cells as a source of inflammatory factors for tumor progression,” Cancer and Metastasis Reviews, vol. 29, no. 2, pp.
[144] H. Takamori, Z. G. Oades, R. C. Hoch, M. Burger, and I. U. Schraufstatter, “Autocrine growth effect of IL-8 and GROalpha on a human pancreatic cancer cell line, Capan-1,” Pancreas, vol. 21, no. 1, pp. 52-56, 2000.
[145] K. Lang, B. Niggemann, K. S. Zanker, and F. Entschladen, “Signal processing in migrating T24 human bladder carcinoma cells: Role of the autocrine interleukin-8 loop,” International Journal of Cancer, vol. 99, no. 5, pp. 673-680, 2002.
[146] K. F. Budden, S. L. Gellatly, D. L. A. Wood et al., “Emerging pathogenic links between microbiota and the gut-lung axis,” Nature Reviews Microbiology, vol. 15, no. 1, pp. 55-63, 2017.
[147] G. B. Huffnagle, “The microbiota and allergies/asthma,” PLoS Pathogens, vol. 6, no. 5, pp. 1-3, 2010.
[148] M. F. Neurath, S. Finotto, and L. H. Glimcher, “The role of TH1/TH2 polarization in mucosal immunity,” Nature Medicine, vol. 8, no. 6, pp. 567-573, 2002.
[149] T. B. Clarke, K. M. Davis, E. S. Lysenko, A. Y. Zhou, Y. Yu, and J. N. Weiser, “Recognition of peptidoglycan from the microbiota byNOD1 enhances systemic innate immunity,” Nature Medicine, vol. 16, no. 2, pp. 228-231, 2010.
[149] B. Boursi, R. Mamtani, K. Haynes, and Y.-X. Yang, “Recurrent antibiotic exposure may promote cancer formation-Another step in understanding the role of the human microbiota?”European Journal of Cancer, vol. 51, no. 17, pp. 2655-2664, 2015.
[151] K. Yoda, F. He, K. Miyazawa, M. Kawase, A. Kubota, and M. Hiramatsu, “Orally administered heat-killed Lactobacillus gasseri TMC0356 alters respiratory immune responses and intestinal microbiota of diet-induced obese mice,” Journal of Applied Microbiology, vol. 113, no. 1, pp. 155-162, 2012.
[152] FAO/WHO, “Guidelines for the Evaluation of Probiotics,” 2002.
[153] A. Vasquez, G. Molin, B. Pettersson, M. Antonsson, and S. Ahrne, “DNA-based classification and sequence heterogeneities in the 16S rRNA genes of Lactobacillus casei/paracasei and related species,” Systematic and Applied Microbiology, vol. 28, no. 5, pp. 430-441, 2005.

The invention claimed is:

1. A method for delivering beneficial microbes into the gut-lung and gut-brain axes by converting said microbes into specific agonist/antigen-presenting cells that interact with mucosal therapeutic target receptors, comprising:
Genetically transforming microbes to express DNA sequences encoding one or more agonists of mucosal cell receptors, including chimeric sequences, wherein the agonists are anchored in the microbial cellular membrane and expressed on the extracellular surface as transmembrane proteins, without secretion, such that the agonists bind to their mucosal target receptors while remaining transmembraneously attached to the microbes;
Culturing and amplifying the transformed microbes to obtain a sufficient quantity for administration;
Formulating the transformed microbes into pharmaceutical compositions for disease prevention and treatment, including:
Oral formulations, produced according to probiotic manufacturing standards;
Suppository formulations, for rectal and vaginal administration;
Administering the pharmaceutical compositions at dosages tailored to patient demographics:
Oral and suppository formulations: approximately 5-10 billion colony-forming units per day for children under 12 years of age, and 10-20 billion colony-forming units per day for adolescents and adults;
Providing treatment for durations ranging from a single preventive dose to sustained administration until effective disease resolution.

2. The method of claim 1, wherein the microbes are genetically transformed to express DNA sequences encoding multiple antigens of a single infectious disease agent simultaneously, ensuring that all encoded antigens are anchored in the microbial cellular membrane and expressed on the extracellular surface as transmembrane proteins without secretion.

3. The method of claim 1, wherein the microbes are genetically transformed to express DNA sequences encoding multiple antigens of multiple infectious disease agents simultaneously, ensuring that all encoded antigens are anchored in the microbial cellular membrane and expressed on the extracellular surface as transmembrane proteins without secretion.

4. The method of claim 1, wherein the microbes are genetically transformed to express DNA sequences encoding multiple antigens of multiple non-infectious disease agents simultaneously, ensuring that all encoded antigens are anchored in the microbial cellular membrane and expressed on the extracellular surface as transmembrane proteins without secretion.

5. The method of claim 1, wherein the microbes are genetically transformed to express DNA sequences encoding an antigen(s) of a non-infectious disease agent(s) and an antigen(s) of an infectious disease agent simultaneously, ensuring that all encoded antigens are anchored in the microbial cellular membrane and expressed on the extracellular surface as transmembrane proteins without secretion.

6. The method of claim 1, wherein the microbes are genetically transformed to express DNA sequences encoding chimeric antigens of infectious disease agent(s) and non-infectious disease agent(s) simultaneously, ensuring that all encoded antigens are anchored in the microbial cellular membrane and expressed on the extracellular surface as transmembrane proteins without secretion.

7. A method for converting microbes into specific antigen-presenting cells for the mucosal immune complex, comprising:
Genetically transforming microbes to express DNA sequences encoding one or more antigens from disease agents, including chimeric sequences, wherein the antigens are anchored in the microbial cellular membrane and expressed on the extracellular surface as transmembrane proteins, without secretion, such that the antigens bind to their mucosal targets while remaining transmembraneously attached to the microbes;
Culturing and amplifying the transformed microbes to obtain a sufficient quantity for administration;
Formulating the transformed microbes into pharmaceutical compositions for disease prevention and treatment, including:
Oral formulations, produced according to probiotic manufacturing standards;
Suppository formulations, for rectal and vaginal administration;
Aerosol formulations, for pulmonary delivery;
Topical formulations, for application to accessible mucosal surfaces;
Injectable formulation for subdermal injection
Administering the pharmaceutical compositions at dosages tailored to patient demographics:
Oral and suppository formulations: approximately 5-10 billion colony-forming units per day for children under 12 years of age, and 10-20 billion colony-forming units per day for adolescents and adults;
Aerosol formulation: approximately 1-10 colony-forming units per day;
Topical formulation: approximately 1-5 colony-forming units per day;
Injectable formulation: approximately 0.25-5 colony-forming units per day;
Providing treatment for durations ranging from a single preventive dose to sustained administration until effective disease resolution.

8. The method of claim 7, wherein the microbes are genetically transformed to express DNA sequences encoding multiple antigens of a single infectious disease agent simultaneously, ensuring that all encoded antigens are anchored in the microbial cellular membrane and expressed on the extracellular surface as transmembrane proteins without secretion.

9. The method of claim 7, wherein the microbes are genetically transformed to express DNA sequences encoding multiple antigens of multiple infectious disease agents simultaneously, ensuring that all encoded antigens are anchored in the microbial cellular membrane and expressed on the extracellular surface as transmembrane proteins without secretion.

10. The method of claim 7, wherein the microbes are genetically transformed to express DNA sequences encoding multiple antigens of multiple non-infectious disease agents simultaneously, ensuring that all encoded antigens are anchored in the microbial cellular membrane and expressed on the extracellular surface as transmembrane proteins without secretion.

11. The method of claim 7, wherein the microbes are genetically transformed to express DNA sequences encoding an antigen(s) of a non-infectious disease agent(s) and an antigen(s) of an infectious disease agent simultaneously, ensuring that all encoded antigens are anchored in the microbial cellular membrane and expressed on the extracellular surface as transmembrane proteins without secretion.

12. The method of claim 7, wherein the microbes are genetically transformed to express DNA sequences encoding a library of antigens of infectious disease agent(s) or non-infectious disease agent(s), ensuring that all encoded antigens are anchored in the microbial cellular membrane and expressed on the extracellular surface as transmembrane proteins without secretion.

13. A method for converting microbes into specific agonist-presenting cells for mucosal therapeutic target receptors, comprising:
Genetically transforming microbes to express DNA sequences encoding one or more agonists of mucosal cell receptors, including chimeric sequences, wherein the agonists are anchored in the microbial cellular membrane and expressed on the extracellular surface as transmembrane proteins, without secretion, such that the agonists bind to their mucosal target receptors while remaining transmembraneously attached to the microbes;
Culturing and amplifying the transformed microbes to obtain a sufficient quantity for administration;
Formulating the transformed microbes into pharmaceutical compositions for disease prevention and treatment, including:
Oral formulations, produced according to probiotic manufacturing standards;
Suppository formulations, for rectal and vaginal administration;
Aerosol formulations, for pulmonary delivery;
Topical formulations, for application to accessible mucosal surfaces;
Injectable formulation for subdermal injection;
Administering the pharmaceutical compositions at dosages tailored to patient demographics:
Oral and suppository formulations: approximately 5-10 billion colony-forming units per day for children under 12 years of age, and 10-20 billion colony-forming units per day for adolescents and adults;
Aerosol formulation: approximately 1-10 colony-forming units per day;
Topical formulation: approximately 1-5 colony-forming units per day;
Injectable formulation: approximately 0.25-5 colony-forming units per day;
Providing treatment for durations ranging from a single preventive dose to sustained administration until effective disease resolution.

14. The method of claim 13, wherein the microbes are genetically transformed to express DNA sequences encoding an antagonist(s) of a therapeutic target receptor(s), ensuring that the encoded antagonist(s) is anchored in the microbial cellular membrane and expressed on the extracellular surface as a transmembrane protein without secretion.

15. The method of claim 13, wherein the microbes are genetically transformed to express DNA sequences encoding multiple agonists of a single therapeutic target receptor, ensuring that all encoded peptides are anchored in the microbial cellular membrane and expressed on the extracellular surface as transmembrane proteins without secretion.

16. The method of claim 13, wherein the microbes are genetically transformed to express DNA sequences encoding an agonist(s) and antagonist(s) of multiple therapeutic targets simultaneously, ensuring that all encoded peptides are anchored in the microbial cellular membrane and expressed on the extracellular surface as transmembrane proteins without secretion.

17. The method of claim 13, wherein the microbes are genetically transformed to express DNA sequences encoding an agonist(s) of a therapeutic target receptor(s) and an antigen(s) of an infectious disease agent(s) simultaneously, ensuring that all encoded peptides are anchored in the microbial cellular membrane and expressed on the extracellular surface as transmembrane proteins without secretion.

18. The method of claim 13, wherein the microbes are genetically transformed to express DNA sequences encoding an antagonist(s) for a therapeutic target receptor(s) and an antigen(s) of an infectious disease agent(s) simultaneously, ensuring that all encoded peptides are anchored in the microbial cellular membrane and expressed on the extracellular surface as transmembrane proteins without secretion.

19. The method of claim 13, wherein the microbes are genetically transformed to express DNA sequences encoding an agonist(s) and antagonist(s) for multiple therapeutic target receptor(s) and an antigen(s) of an infectious disease agent(s) simultaneously, ensuring that all encoded peptides are anchored in the microbial cellular membrane and expressed on the extracellular surface as transmembrane proteins without secretion.

20. The method of claim 13, wherein the microbes are genetically transformed to express DNA sequences encoding a library of agonist(s) or antagonist(s) for multiple therapeutic target receptors, ensuring that all encoded peptides are anchored in the microbial cellular membrane and expressed on the extracellular surface as transmembrane proteins without secretion.
